(19) United States
(12) Patent Application Publication
(10) Pub. No.: US 2018/0064850 A1
(43) Pub. Date: Mar. 8, 2018

(54) Biocompatible implants for use in tendon therapy

(71) Applicant: The University Court of the University of Glasgow, Glasgow (GB)

(72) Inventors: Derek Stewart Gilchrist, Glasgow (GB); Neal Lindsay Millar, Glasgow Strathclyde (GB)

(73) Assignee: The University Court of the University of Glasgow, Glasgow (GB)

(21) Appl. No.: 15/557,050

(22) PCT Filed: Mar. 9, 2016
(86) PCT No.: PCT/GB2016/050638

§ 371 Date: Sep. 8, 2017

(30) Foreign Application Priority Data
Mar. 9, 2015 (GB) 1503967.0

(51) Int. Cl. A61L 27/36; A61L 27/38; A61L 27/54; A61L 27/56

(52) U.S. Cl. CPC A61L 27/3662; A61L 27/3633; A61L 27/3834; A61L 2300/258; A61L 27/54; A61L 27/56; A61L 2430/10; A61L 27/386

(57) ABSTRACT

The invention provides biocompatible implants (“scaffolds”) for use in the treatment of tendon injury and/or modulation of the biomechanical properties of tendon. More particularly, the invention provides biocompatible implants capable of delivering microRNA 29 and precursors and mimics thereof to the tendon. In some embodiments the implant comprises a bioresorbable substrate to avoid the need for surgical removal of the implant once healing or re-modelling is complete.

### FIELD OF THE INVENTION

The invention relates to biocompatible implants, and in particular to their use for delivery of microRNA 29 and precursors and mimics thereof for the treatment of tendon injury and/or modulation of the biomechanical properties of tendon.

### BACKGROUND TO THE INVENTION

Dysregulated tissue repair and inflammation characterise many common musculoskeletal pathologies′, including tendon disorders. Tendinopathies represent a common precipitant for musculoskeletal consultation in primary care²⁻³ and comprise 30-50% of all sports injuries³. Tendinopathy is characterised by altered collagen production from subtype 1 to 3 resulting in a decrease in tensile strength that can presage clinical tendon rupture⁴.

Inflammatory mediators are considered crucial to the onset and perpetuation of tendinopathy³. Expression of various cytokines has been demonstrated in inflammatory cell lineages and tenocytes suggesting that both infiltrating and resident populations participate in pathology⁶⁻⁹. Mechanical properties of healing tendons in IL-6-deficient mice are inferior compared with normal controls' while TNF-α blockade improves the strength of tendon-bone healing in a rat tendon injury model¹¹. While these data raise the intriguing possibility that cytokine targeting could offer therapeutic utility, there is currently insufficient mechanistic understanding of cytokine/matrix biology in tendon diseases to manifest this possibility in practice.

Cytokines are often regulated at the post-transcriptional level by microRNA (miRNA); small non-coding RNAs that control gene expression by translational suppression and destabilization of target mRNAs¹². microRNA networks are emerging as key homeostatic regulators of tissue repair with fundamental roles proposed in stem cell biology, inflammation, hypoxia-response, and angiogenesis¹³.

Tissue engineering techniques offer significant potential to enhance and accelerate tendon injury repair. Biocompatible implants, often referred to as “scaffolds”, have been proposed for use in stabilising and supporting the injury site during healing, providing substrates for cell growth during the repair process, and delivery of active molecules such as growth factors which stimulate appropriate cell growth and migration. Use of bioresorbable materials enables the scaffold to be incorporated into and absorbed by the repaired tissue, with no requirement for removal later.

### SUMMARY OF THE INVENTION

Healing of tendon injury is often sub-optimal, at least in part due to a shift in collagen synthesis from type 1 to type 3 during tendinopathy. Type 3 collagen is mechanically inferior to type 1 collagen, resulting in a tendon with lower tensile strength. The biomechanical properties of the tendon would be improved if the balance between the collagen subtypes could be modulated back towards type 1 collagen.

miR-29 has been previously identified as a regulator of collagen synthesis in various biological processes, such as fibrosis and scleroderma. However, the inventors have found, for the first time, that tenocytes contain alternatively spliced forms of type 1 collagen transcripts. The predominant transcripts for type 1a1 and 1a2 collagen have short 3′ untranslated regions (UTRs) which do not contain miR-29 binding sites, while the overwhelming type 3 collagen transcript present is a long miR-29-sensitive form.

As a result, synthesis of type 1 collagen in tenocytes is affected to a much lesser degree by miR-29 than synthesis of type 3 collagen. Surprisingly, then, by up-regulating miR-29 activity, it is possible to modulate the balance between the collagen subtypes in favour of type 1 collagen, thus mitigating or abrogating the reduction in tensile strength of the tendon and modulating its biomechanical properties such as its ultimate failure strength.

In its broadest form, the invention relates to a biocompatible implant for use in a method of tendon therapy, wherein the implant is capable of delivering miR-29, a mimic thereof, or a precursor of either, to the tendon, e.g. at a site of injury.

Thus the invention provides a biocompatible implant comprising

(a) a biocompatible substrate capable of supporting growth of tendon cells; and
(b) a modulator of tendon healing;
wherein said modulator is
(i) miR-29, a mimic thereof, or a precursor of either; or
(ii) a nucleic acid encoding miR-29, a mimic thereof, or a precursor of either;
and wherein said modulator is located extracellularly to any cells present on or in said substrate.

The invention also provides a biocompatible implant as described above for use in a method of tendon therapy, e.g. in a method of surgery performed on a subject in need thereof.

The invention further provides a method of tendon therapy comprising locating a biocompatible implant as described above at a site of injury.

The invention also provides the use of a modulator of tendon healing in the preparation of a biocompatible implant for use in a method of tendon therapy, wherein said implant comprises

(a) a biocompatible substrate capable of supporting growth of tendon cells; and
(b) said modulator of tendon healing;
wherein said modulator is
(i) miR-29, a mimic thereof, or a precursor of either; or
(ii) a nucleic acid encoding miR-29, a mimic thereof, or a precursor of either.

As set out above, the modulator is located extracellularly to any cells present on or in said substrate.

In certain embodiments, the modulator is incorporated into the substrate before the implant is introduced to the target site. In other embodiments, the substrate may be introduced to the target site and the modulator subsequently applied to the substrate in situ, e.g. as part of the same surgical procedure.

Thus the invention also provides a modulator of tendon healing for use in a method of tendon therapy;

wherein said method comprises applying said modulator to a biocompatible substrate capable of supporting growth of tendon cells;
wherein said biocompatible substrate is located at a site of tendon injury;
and wherein said modulator is:
(i) miR-29, a mimic thereof, or a precursor of either; or
(ii) a nucleic acid encoding miR-29, a mimic thereof, or a precursor of either.

The invention also provides the use of a modulator of tendon healing in the preparation of a pharmaceutically acceptable composition;

wherein said composition is for use in a method of tendon therapy which comprises applying said composition to a biocompatible substrate capable of supporting growth of tendon cells;
wherein said biocompatible substrate is located at a site of tendon injury;
and wherein said modulator is
(i) miR-29, a mimic thereof, or a precursor of either; or
(ii) a nucleic acid encoding miR-29, a mimic thereof, or a precursor of either.

Also provided is a method of tendon therapy comprising locating a biocompatible substrate capable of supporting growth of tendon cells at a site of tendon injury, and applying a modulator of tendon healing to the biocompatible substrate,

wherein said modulator is
(i) miR-29, a mimic thereof, or a precursor of either; or
(ii) a nucleic acid encoding miR-29, a mimic thereof, or a precursor of either.

Also provided is a kit comprising (a) a biocompatible substrate capable of supporting growth of tendon cells, and (b) a modulator of tendon healing, wherein said modulator is:

(i) miR-29, a mimic thereof, or a precursor of either; or
(ii) a nucleic acid encoding miR-29, a mimic thereof, or a precursor of either.

The implant typically provides a conducive environment for adhesion, replication and migration of tendon cells, and thus for repair and remodelling of the tendon tissue. Thus, the substrate will typically be infiltrated by host cells and/or by exogenously seeded cells and incorporated into the structure of the repaired tendon. Such an implant is often referred to as a “scaffold”. The implant may also provide mechanical support to off-load any lesion during the healing process. In its broadest form, the term “capable of supporting growth of tendon cells” means that the substrate is not toxic to tendon cells in contact with it, and preferably does not inhibit replication or migration of tendon cells in contact with it. Preferably, tendon cells are capable of adhering to the substrate, replicating while in contact with the substrate, and/or migrating across it.

The substrate is composed of biocompatible materials and is preferably bioresorbable, i.e. composed of materials which can be broken down within the body, to reduce or eliminate the need for mechanical (i.e. surgical) removal of the implant once healing is complete.

The substrate may comprise one or more cells. Suitable cells include tendon cells (e.g. tenocytes or tenoblasts) and precursors thereof (e.g. mesenchymal stem cells).

One or more cells may be applied to the substrate prior to introduction of the substrate at the target site. Alternatively, one or more cells may be applied to the substrate after location of the substrate at the target site.

Thus the invention extends to a method of preparing an implant of the invention comprising providing a substrate as described herein, contacting said substrate with a tendon cell or a precursor thereof, and culturing the substrate. Such methods enable the production of a cellularised or partially cellularised implant in vitro or ex vivo and may assist in the formation of appropriate ECM before introduction of the implant to the recipient.

The substrate may be porous. For example, it may comprise a fabric of woven or unwoven fibres. Alternatively the substrate may comprise a matrix or foam. For example, the substrate may comprise a gel, such as a hydrogel.

The mean pore diameter may be in the range of 10-500 μm, e.g. 50-500 μm, e.g. 100-500 μm or 200-500 μm.

The substrate may comprise or consist of extra-cellular matrix (ECM).

The ECM may be derived from a tissue explant, e.g. from connective tissue (such as tendon), small intestinal submucosa (SIS), dermis or pericardium, or may have been generated by cell culture.

Preparation of the ECM for use as a substrate may comprise a step of decellularisation (e.g. by treatment with an appropriate protease, such as trypsin), a step of oxidation (e.g. with peracetic acid), a step of freeze drying, or any combination thereof.

Additionally or alternatively, preparation of the ECM may comprise a step of chemical cross-linking.

The resulting ECM may be sterilized prior to use.

The ECM may be re-hydrated prior to implantation, e.g. with an aqueous solution, which may be any physiologically compatible or pharmaceutically acceptable solution, such as physiological saline solution or PBS.

Alternatively, the substrate may be a synthetic substrate, e.g. a substrate formed other than by biological cells. A synthetic substrate may nevertheless comprise biological components (i.e. components which occur in nature) such as proteins, polysaccharides and other biological polymers, as well as synthetic components (i.e. components which do not occur in nature) such as synthetic polymers.

Thus the substrate may comprise one or more proteins or polysaccharides. Suitable proteins include collagen, elastin, fibrin, albumin and gelatin. Suitable polysaccharides include hyaluronan, alginate and chitosan. Many of these, such as collagen, elastin and hyaluronan are natural components of the extracellular matrix.

Suitable synthetic components include biocompatible synthetic polymers, such as polyvinyl alcohol, oligo[poly(ethylene glycol) fumarate] (OPF), and polymers and co-polymers of monomers such as glycolic acid and lactic acid, such as poly(glycolic acid) (PGA), poly(lactic acid) (PLA) and poly(lactic-co-glycolic acid) (PLGA).

Additionally or alternatively, the substrate may comprise or consist of a bioceramic material, such as hydroxyl carbonate apatite (HCA) or tricalcium phosphate, or a biodegradable metallic material, such as porous magnesium or magnesium oxide.

The substrate may be composed of a plurality of layers, for example it may comprise a plurality of layers of fabric or ECM. The substrate may comprise a gradient structure, mimicking the transition from collagen to bone at the enthesis. The gradient may represent increasing hardness and/or increasing mineralisation (calcification), e.g. as described in references 47 and 48.

Even when the substrate is not principally composed of extracellular matrix, it may nevertheless be desirable that the substrate comprises some proportion of one or more extracellular matrix components, such as collagen, elastin, hyaluronan, etc.

The substrate may further comprise one or more cell adhesion peptides to promote cell adhesion. A cell adhesion peptide may comprise or consist of an integrin binding motifs or a heparin binding motif.

The substrate may further comprise one or more extracellular growth factors, e.g. bFGF (basic fibroblast growth factor, also designated FGF2 or FGF-beta) and TGF-beta (transforming growth factor beta).

In any aspect, the miR-29 which constitutes the modulator, or which is encoded by the modulator, may be miR-29a, miR-29b (b1 and/or b2), miR-29c or any combination thereof. It may be desirable that the miR-29 is miR-29a or a combination including miR-29a.

If desired, the modulator may be provided in association with (e.g. complexed with or encapsulated by) a suitable carrier molecule, such as a pharmaceutically acceptable lipid or polymer or a combination thereof.

The carrier molecule may further comprise a targeting agent capable of binding to the surface of the target cell.

Where the modulator is a nucleic acid encoding miR-29, a mimic thereof, or a precursor of either, it may be provided as part of a viral vector. The viral vector may, for example, be an adenovirus, adeno-associated virus (AAV), retrovirus (especially lentivirus) or herpesvirus vector.

Other features of the modulator are described in more detail below.

### MicroRNA

MicroRNAs (miRs) are small non-coding RNAs that have a substantial impact on cellular function through repression of translation (either through inhibition of translation or induction of mRNA degradation). MicroRNAs derive from primary RNA transcripts (pri-miRNA) synthesised by RNA pol II, which may be several thousand nucleotides in length. A single pri-miRNA transcript may give rise to more than one active miRNA.

In the nucleus, the Type III RNAse enzyme Drosha processes the pri-miRNA transcript into a precursor miRNA (pre-miRNA) consisting of a stem-loop or hairpin structure, normally around 70 to 100 nucleotides in length. The pre-miRNA is then transported to the cytoplasm, where it is processed further by the RNAse Dicer, removing the loop and yielding a mature double stranded miRNA molecule, having an active “guide” strand (typically 15 to 25 nucleotides in length) hybridised to a wholly or partially complementary “passenger” strand.

The mature double stranded miRNA is then incorporated into the RNA-induced silencing complex, where the guide strand hybridises to a binding site in the target mRNA.

The guide strand may not be completely complementary to the target binding site. However, a region of the guide strand designated the “seed” sequence is usually fully complementary to the corresponding sequence of the target binding site. The seed sequence is typically 2 to 8 nucleotides in length and located at or near (within 1 or two nucleotides of) the 5′ end of the guide strand.

It is believed that single unpaired guide strands may also be capable of being incorporated into RISC. It is also believed that modifications to the passenger strand (e.g. to the sugars, the bases, or the backbone structure) which impede incorporation of the passenger strand into RISC may also increase efficiency of target inhibition by a double stranded miRNA.

miR-29 and Precursors Thereof

In the present invention, the modulator of tendon healing is:

(i) miR-29, a mimic thereof, or a precursor of either; or

(ii) a nucleic acid encoding miR-29, a mimic thereof, or a precursor of either.

The three main isoforms of miR-29 in humans are miR-29a, miR-29b1, miR-29b2, and miR-29c.

The term “miR-29” is used in this specification to refer to an RNA oligonucleotide consisting of the mature “guide strand” sequence of any one of these three isoforms.

Mature human miR-29a (“hsa-miR-29a”) has the sequence:

UAGCACCAUCUGAAAUCGGUUA.

Mature miR-29b1 and miR-29b2 (“hsa-miR-29b1” and “hsa-miR-29b2”) are identical and have the sequence:

UAGCACCAUUUGAAAUCAGUGUU.

Mature human miR-29c (“hsa-miR-29c”) has the sequence:

UAGCACCAUUUGAAAUCGGUUA.

It is conventional in micro-RNA naming to include a three letter prefix designating the species from which the micro-RNA originates. Thus “hsa” stands for Homo sapiens. These mature miR29 sequences are found identically in most mammals, including horse.

All four mature guide strands share the same “seed” region, which binds to the target mRNA, and has the sequence:

AGCACCA.

The miR-29 guide strand oligonucleotide may be single stranded, or it may be hybridised with a second RNA oligonucleotide, referred to as a “passenger strand”. The guide strand and passenger strand run anti-parallel to one another in the hybridised complex, which may be referred to as “double stranded miR-29”. (The guide strand, when present in isolation, may be referred to as “single stranded miR-29”.)

The passenger strand and the guide strand may contain a number of mis-matches with the result that not all nucleotides in one or both strands hybridise to complementary nucleotides in the other strand. Thus the double stranded miR-96 may contain one or more bulges (a bulge is an unpaired nucleotide, or plurality of consecutive unpaired nucleotides, in one strand only) or internal loops (opposed unpaired nucleotides in both strands). One or more nucleotides at the termini may also be unpaired.

The passenger strand may be 100% complementary to the seed sequence of the guide strand.

The native human passenger strands have the sequence:

(miR29a)
ACUGAUUUCUUUUGGUGUUCAG
(miR-29b1)
GCUGGUUUCAUAUGGUGGUUUAGA;
(miR-29b2)
CUGGUUUCACAUGGUGGCUUAG;
and
(miR-29c)
UGACCGAUUUCUCCUGGUGUUC.

One or both strands of double stranded miR-29 may comprise a 3′ overhang, e.g. of 1, 2 or 3 nucleotides. That is to say, one or two nucleotides at the 3′ terminus of the strand extend beyond the most 5′ nucleotide of the complementary strand (including any unpaired terminal nucleotides) and thus have no corresponding nucleotides in the complementary strand. For example, both strands may comprise a 3′ overhang of 1, 2 or 3 nucleotides. Alternatively the complex may be blunt-ended at one or both ends. In some embodiments, the passenger strand is the same length as the guide strand, or differs in length, e.g. by up to five nucleotides or even more, depending on the degree of mismatch between the two strands and the lengths of any 3′ overhang.

Precursors of miR-29 include pre-mir-29 and pri-mir-29 of any of the three isoforms, as well as fragments and variants thereof which can be processed to mature miR-29 (whether single or double stranded).

The term “pre-mir-29” is used to refer to an RNA oligonucleotide consisting of any full-length mammalian pre-mir-29 sequence, or a fragment or variant thereof which comprises a mature miR-29 guide sequence connected by a loop sequence to a corresponding passenger sequence which is fully or partially complementary to the guide sequence, and wherein the oligonucleotide is capable of forming a stem-loop structure (or “hairpin”) in which the guide sequence and passenger sequence hybridise to one another.

A pre-mir-29 is capable of acting as a substrate for the double-stranded RNA-specific ribonuclease (RNAse III-type enzyme) Dicer, whereby it is processed to a mature double stranded miR-29.

Full-length mammalian pre-mir-29 sequences include the human sequences:

AUGACUGAUUUCUUUUGGUGUUCAGAGUCAAUAUAAUUUUCUAGCACCAU
CUGAAAUCGGUUAU (hsa-pre-mir-29a: alternative
(i));
AUGACUGAUUUCUUUUGGUGUUCAGAGUCAAUAUAAUUUUCUAGCACCAU
CUGAAAUCGGUUAU AAUGAUUGGGG (hsa-pre-mir-29a:
alternative (ii));
CUUCAGGAAGCUGGUUUCAUAUGGUGGUUUAGAUUUAAAUAGUGAUUGUC
UAGCACCAUUUGAAAUCAGUGUUCUUGGGGG (hsa-pre-mir-
29b1);
CUUCUGGAAGCUGGUUUCACAUGGUGGCUUAGAUUUUUCCAUCUUUGUAU
CUAGCACCAUUUGAAAUCAGUGUUUUAGGAG (hsa-pre-mir-
29b2);
and
AUCUCUUACACAGGCUGACCGAUUUCUCCUGGUGUUCAGAGUCUGUUUUU
GUCUAGCACCAUUUGAAAUCGGUUAUGAUGUAGGGGGA (hsa-pre-
mir-29c)

The corresponding mature guide strand sequences are underlined.

The pre-mir-29 may possess one or more modifications outside the mature sequence, compared to the sequences shown.

The sequence upstream (5′) of the mature sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

For example, the sequence upstream (5′) of the miR-29a mature sequence may differ by up to 20 nucleotides from the corresponding 5′ human sequence when optimally aligned therewith, e.g. by 1, 2, 3, 4, 5, 6, 7, 8, 9, 10, 11, 12, 13, 14, 15, 16, 17, 18, 19 or 20 nucleotides.

The sequence upstream of the miR-29b1 or b2 mature sequence may differ by up to 25 nucleotides from the corresponding 5′ human sequence when optimally aligned therewith, e.g. by 1, 2, 3, 4, 5, 6, 7, 8, 9, 10, 11, 12, 13, 14, 15, 16, 17, 18, 19, 20, 21, 22, 23, 24 or 25 nucleotides.

The sequence upstream of the miR-29c mature sequence may differ by up to 25 nucleotides from the corresponding 5′ human sequence when optimally aligned therewith, e.g. by 1, 2, 3, 4, 5, 6, 7, 8, 9, 10, 11, 12, 13, 14, 15, 16, 17, 18, 19, 20, 21, 22, 23, 24 or 25 nucleotides.

The sequence downstream (3′) of the mature sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

The sequence downstream (3′) of the miR-29a mature sequence may be the same as the 3′ human sequence, or may be different. It may be a different nucleotide from that found in the shorter of the two sequences shown above, i.e. alternative (i). It may be longer than the sequence shown in alternative (i). For example, it may differ by up to 6 nucleotides from the corresponding 3′ sequence of alternative (ii) shown above.

The sequence downstream (3′) of the miR-29b1 or b2 mature sequence may differ by up to 4 nucleotides from the corresponding 3′ human sequence when optimally aligned therewith, e.g. by 1, 2, 3 or 4 nucleotides.

The sequence downstream (3′) of the miR-29c mature sequence may differ by up to 7 nucleotides from the corresponding 3′ human sequence when optimally aligned therewith, e.g. by 1, 2, 3, 4, 5, 6 or 7 nucleotides.

The term “pri-mir-29” is used to refer to an RNA oligonucleotide consisting of any full-length mammalian pri-mir-29 sequence, or a fragment or variant thereof which comprises a pre-mir-29 sequence and is capable of being processed to a pre-mir-29 sequence by the double-stranded RNA-specific ribonuclease (RNAse III-type enzyme) Drosha.

A single transcript may be capable of being processed into two or more mir-29 molecules, mimics or precursors thereof.

hsa-mir29a and mir29b1 are encoded in the final exon of the transcript having GenBank Accession Number EU154353 (EU154353.1 GI:161824377). The region encoding mir29a and mir29b1, plus flanking sequence, is shown below. (Hsa-mir29a is shown in bold upper case font with mature miR-29a sequence being underlined. Hsa-mir29b is shown in upper case font with miR-29b being underlined.)

gaaagcguuu uucuucaacu ucuauggagc acuugcuugc
uuuguccuau uugcaugucc gacggacggu ucuccagcac
cacugcuagu cguccuccgc cugccugggu acuugaucac
aggaugccuc ugacuucucc ugccuuuacc caagcaaagg
auuuuccuug ucuucccacc caagagugac ggggcugaca
ugugcccuug ccucuaaaug augaagcuga accuuugucu
gggcaacuua acuuaagaau aagggagucc caggcaugcu
cucccaucaa uaacaaauuc agugacauca guuuaugaau
auaugaaauu ugccaaagcu cuguuuagac cacugaguaa
cucacagcua gguuucaacu uuuccuuucu agguugucuu
ggguuuauug uaagagagca uuaugaagaa aaaaauagau
cauaaagcuu CUUCAGGAAG CUGGUUUCAU AUGGUGGUUU
AGAUUUAAAU AGUGAUUGUC UAGCACCAUU UGAAAUCAGU
GUUCUUGGGG Gagaccagcu gcgcugcacu accaacagca
aaagaaguga augggacagc ucugaaguau uugaaagcaa
cagcaggaug gcugugagaa ccugccucac auguagcuga
ccccuuccuc accccugcca acaguggugg cauauaucac
aaauggcagu caggucucug cacuggcgga uccaacugug
aucgaaaguu uuccaaaaau aaguuguguc uguauugaac
augaacagac uuucuucuug ucauuauucu cuaacaauac
ugcauaacaa uuauuugcau acauuugcau ugcauuaagu
auucuaagua aucuagagac gauuuaaagu auacgggagg
auguguguag guuguaugca aauacuacac cauuuucuau
cagagacuug agcaucugug gauuuuggua uccaaggggc
uuucuggaac caaucccuca aggauaccaa gggaugaaug
uaauuguaca ggauaucgca uuguuggaau uuuauacuuc
uuuguggaau aaaccuauag cacuuaauag auaguacaga
cucauuccau ugugccuggg uuaaagagcc caauguaugc
uggauuuagu aagauuuggg cccucccaac ccucacgacc
uucugugacc CCUUAGAGGA UGACUGAUUU CUUUUGGUGU
UCAGAGUCAA UAUAAUUUUC UAGCACCAUC UGAAAUCGGU
UAUaaugauu gggqaagagc accaugaugc ugacugcuga
gaggaaaugu auuggugacc guuggggcca uggacaagaa
cuaagaaaac aaaugcaaag caauaaugca aaggugauuu
uucuucuucc aguuucuaag uugaauuuca cugaccugaa
uugcaugugg uauaauacua acaaaugguu cacuauuagc
auaucaugaa ugguuauacu uuauagaaau uccauagacu
ugguggqggu uuuquuuugg ugacggauac cuagaaacac
uccuggggaa aaucgaugac uggcuuagau gaugggaaag
gagcagcgag ggagucaauu cuguuguuga ugagaagcug
caccagcuau cucugaacuc uccucucuua gcuggcugag
gaguucccuc caugguuaaa caggucauuu ucuuacauaa
ggaaaaaugg uccagagaaa cuggguuucu auggcugaga
cagaacugug cuaauaugug uc

hsa-pri-miR29b2 and hsa-pri-mir29c are encoded in a single transcript shown below. hsa-mir29b2 is shown upper case font with mature hsa-miR-29b2 underlined. hsa-mir29c is shown in bold upper case font with mature hsa-miR-29c underlined.

agcuuucuaa aaucucuuua ggggugugcg uaggcuccug ugucuaugcc ugcuuuugac
ugcccaguug aagccucuuc cuaugccuuu uaaaauuuca cgcacuauaa ggaggaagag
cucagggcuc ccaaaacuuu uuauuuagag ggaagaaugc uagggagaug gguaugcaga
ggguugacca aauuggaaga aaauauuuau ucuguaguuu gguguuggaa aagggaauuu
uccaaucagc cacaccucag uguugcggca aaauaauucu uggcuccccu ggaaacgcau
gggcaaggua gggcagagcu gcugcugcug auacugccac cacccugggc uuccugcuga
cucugggcua cucccugggg acaacagauu ugcauugacg uccggggcug uccagaggcc
cucaagagcc aguugugagc ugagcccagu augggaaaga ucuaccuucu ggaagcuacu
acuacguggu gcuuggaaag aggacucagg agagugcagc uugcucugug agugggugac
aaccucuugg cgacucaggc ucagcugagg auggugccag ugugccggag acagccguca
uacugccgga uagaguggcu cacuugcaug uauuuggaac aaaaaaagga gaugccuggc
agccccgcuc ucuggagugc uguugagcca ccaauuuuug ugguuuugug accacaagug
cugacugaug cgacaugacc ccagucuugu cagugaauca ucaccaggcu gcuuacugga
aacuggaugc agcaaggaaa uaggauuuaa ccgcucucug ccucccagga gcccugaaau
cagcauuccc agaggaaaga agauggccau cugggcuugg cuuccggcuc cccccaucug
gcuggaacac acaucaguca ccccugugua accuccucug ugccuuuccc auggagcacu
gugucauauc acaaguagaa cuacaagaag auauuucucc ucagggcaga ggcugggucu
uccgauugaa ucucccuucu uucuucauug agauccuCUU CUUCUGGAAG CUGGUUUCAC
AUGGUGGCUU AGAUUUUUCC AUCUUUGUAU CUAGCACCAU UUGAAAUCAG UGUUUUAGGA
Guaagaauug_cagcacagcc aaggguggac ugcagaggaa cugcugcuca uggaacuggc
uccucuccuc uugccacuug agucuguucg agaaguccag ggaagaacuu gaagagcaaa
auacacucuu gaguuuguug gguuuuggga gaggugacag uagagaaggg gguuguguuu
aaaauaaaca caguggcuug agcaggggca gagguuguga ugcuauuucu guugacuccu
agcagccauc accagcauga auguguucgu agggccuuug aguguggcga uugucauauu
cuguuggaua acaauguauu gggugucgau ugucaugggg caggggagag ggcaguacac
cuggaggacc auuuugucca caucgacacc aucagucugc ucuuagagga ugcccuggag
uauucggcgu ugauugcggg gcacccgaaa ucagacuugc caccuggacu gucgaggugc
agacccuggg agcaccacug goccAUCUCU UACACAGGCU GACCGAUUUC UCCUGGUGUU
CAGAGUCUGU UUUUGUCUAG CACCAUUUGA AAUCGGUUAU GAUGUAGGGG GAaaagcagc
agccucgaag ccucaugcca acucugggca gcagcagccu gugguuuccu ggaagaugga
ugggcagaga auagggaagg aagaucaugc uuuucccuac uaacuucugu aacugcaugu
augauacauu auugcagagg uaagagauag uuuaauggau uuuuaaaaac aaauuacuau
aauuuaucug auguucucua guugcauuuu gcugaaaugu agugcuguuc uaaauucugu
aaauugauug cuguugaauu aucuuucugu ugagaagagu cuauucaugc auccugaccu
uaauaaauac uauguucagu uu

Thus a pri-mir-29 may contain more than one mature miR-29 or mimic sequence. For example, it may contain miR-29a and miR-29b1 or mimics thereof, or miR-29b2 and miR-29c or mimics thereof.

Alternatively, the pri-mir-29 may contain just one mature miR-29 sequence of a mimic thereof.

The pri-mir-29 may have at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with either of the pri-mir-29 sequences shown above, or with a fragment of one of those sequences containing one of the mature miR-29 sequences.

The pri-mir-29 may possess one or more modifications outside the mature sequence or outside the native pre-mir-29 sequence, compared to the sequences shown.

For example, the sequence upstream (5′) of the mature sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

The sequence upstream (5′) of the pre-mir-29 sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

The sequence downstream (3′) of the mature sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

The sequence downstream (3′) of the native pre-mir-29 sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

The miR-29 precursor may be any suitable length, as long as it can be processed to mature miR-29 (whether single or double stranded). Thus a miR-29a precursor is at least 23 nucleotides in length, a miR29b precursor is at least 24 nucleotides in length, and a miR-29c precursor is at least 25 nucleotides in length.

The miR29 precursor may be at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 200, at least 250, at least 300, at least 350, at least 400, at least 450, at least 500, at least 1000, at least 1500 or at least 2000 nucleotides in length.

Alternatively, the precursor may be a maximum of 25, 30, 35, 40, 45, 50, 55, 60, 65, 70, 75, 80, 85, 90, 95, 100, 110, 120, 130, 140, 150, 200, 250, 300, 350, 400, 450, 500, 1000, 1500, 2000 or 2500 nucleotides in length, although longer precursor transcripts are possible.

It should be noted that the term “oligonucleotide” is not intended to imply any particular length, and is simply used to refer to any single continuous chain of linked nucleotides.

miR-29 Mimics and Precursors Thereof

A miR-29 mimic is an oligonucleotide which has one or more modifications in structure or sequence compared to naturally-occurring miR-29 but retains the ability to hybridise to a miR-29 binding site in mRNA regulated by miR-29, and to inhibit translation or promote degradation of such an mRNA, e.g. to inhibit production of a protein encoded by that mRNA. mRNAs regulated by miR-29 include type 3 collagen (Col3a1).

Examples of miR-29 binding sites include:

CCAUUUUAUACCAAAGGUGCUAC (from Col1a1 mRNA);
UGUUCAUAAUACAAAGGUGCUAA (from Col1a2 mRNA);
and
UUCAAAAUGUCUCAAUGGUGCUA (from col3a1 mRNA).

A miR-29 mimic oligonucleotide is typically 15-35 nucleotides in length, e.g. 15 to 30, 15 to 25, 18 to 25, 20 to 25, e.g. 20 to 23, e.g. 20, 21, 22 or 23 nucleotides in length.

The miR-29 mimic may differ in base sequence, nucleotide structure, and/or backbone linkage as compared to one of the native miR-29 mature sequences.

The miR-29 mimic comprises a seed sequence which may be identical to the native seed sequence:

AGCACCA

or may differ from the native seed sequence at no more than three positions, e.g. at no more than two positions, e.g. at no more than one position. Preferably the seed sequence is identical to that shown.

The miR-29 mimic may comprise or consist of an oligonucleotide having a mature native miR-29 guide sequence such as:

UAGCACCAUCUGAAAUCGGUUA (hsa-miR-29a);
UAGCACCAUUUGAAAUCAGUGUU (hsa-miR-29b1 and 2);
or
UAGCACCAUUUGAAAUCGGUUA (hsa-miR-29c);

(wherein the seed sequence is underlined in each case);
or which differs from the mature native sequence at:
(i) no more than three positions within the seed sequence; and
(ii) no more than five positions outside the seed sequence.

Thus the mimic seed sequence differs from the native seed sequence at no more than three positions, e.g. at no more than two positions, e.g. at no more than one position. Preferably the seed sequence is identical to the native seed sequence.

Additionally or alternatively, the mimic differs from the native sequence outside the seed sequence at no more than five positions, e.g. at no more than four positions, no more than three positions, no more than two positions, e.g. at no more than one position.

The miR-29 mimic may be hybridised to a second oligonucleotide. As with the native miR-29, the active oligonucleotide may be referred to as the “guide strand” and the associated oligonucleotide as the “passenger strand”. The hybridised complex may be referred to as a double stranded miR-29 mimic.

The sequence of the mimic passenger strand may be identical to the sequence of the native passenger strand or may differ from the native passenger strand at one or more positions. For example, the sequence of the mimic passenger strand may differ from that of the native passenger strand at no more than 10 positions, no more than 9 positions, no more than 8 positions, no more than 7 positions, no more than 6 positions, no more than 5 positions, no more than 4 positions, no more than 3 positions, no more than 2 positions or no more than 1 position.

One or both strands of a double stranded miR-29 mimic may comprise a 3′ overhang of 1 or 2 nucleotides. For example, both strands may comprise a 3′ overhang of 2 nucleotides. Alternatively the complex may be blunt-ended at one or both ends. In some embodiments, the passenger strand is the same length as the guide strand, or differs in length by one or two nucleotides.

A precursor of a miR-29 mimic is any molecule which can be processed within the target cell to a miR-29 mimic as defined above, typically by action of the enzyme Dicer or by sequential action of the enzymes Drosha and Dicer.

Thus a precursor may have additional oligonucleotide sequence upstream (5′) and/or downstream (3′) of the mimic sequence.

The precursor may comprise the miR-29 mimic guide sequence connected by a loop sequence to a corresponding passenger sequence which is fully or partially complementary to the guide sequence, and wherein the oligonucleotide is capable of forming a stem-loop structure (or “hairpin”) in which the guide sequence and passenger sequence hybridise to one another. Such an oligonucleotide may be regarded as a pre-mir-29 mimic and is capable of acting as a substrate for the double-stranded RNA-specific ribonuclease (RNAse III-type enzyme) Dicer, whereby it is processed to a double stranded miR-29 mimic, comprising separate guide and passenger strands.

The sequence upstream (5′) of the mature sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

The sequence downstream (3′) of the mature sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

Alternatively, the precursor may be a pri-mir-29 mimic (i.e. it has additional oligonucleotide sequence upstream (5′) and/or downstream (3′) of the pre-mir-29 mimic sequence) and be capable of being processed to a pre-mir-29 mimic sequence by the double-stranded RNA-specific ribonuclease (RNAse III-type enzyme) Drosha.

For example, the sequence upstream (5′) of the mature miR-29 mimic sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

The sequence upstream (5′) of the pre-mir-29 mimic sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

The sequence downstream (3′) of the mature miR-29 mimic sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

The sequence downstream (3′) of the pre-mir-29 mimic sequence may have, for example, at least 50% identity, at least 55% identity, at least 60% identity, at least 65% identity, at least 70% identity, at least 75% identity, at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity with the corresponding human sequence.

The miR-29 mimic precursor may be any suitable length, as long as it can be processed to mature miR-29 mimic (whether single or double stranded). Thus the precursor is at least 23 nucleotides in length, and may be at least 25, at least 30, at least 35, at least 40, at least 45, at least 50, at least 55, at least 60, at least 65, at least 70, at least 75, at least 80, at least 85, at least 90, at least 95, at least 100, at least 110, at least 120, at least 130, at least 140, at least 150, at least 200, at least 250, at least 300, at least 350, at least 400, at least 450, at least 500, at least 1000, at least 1500 or at least 2000 nucleotides in length.

Alternatively, the precursor may be a maximum of 25, 30, 35, 40, 45, 50, 55, 60, 65, 70, 75, 80, 85, 90, 95, 100, 110, 120, 130, 140, 150, 200, 250, 300, 350, 400, 450, 500, 1000, 1500, 2000 or 2500 nucleotides in length.

### Structural Modifications

In addition to, or as an alternative to the sequence modifications discussed above, a miR-29 mimic or precursor thereof may comprise one or more structural modifications compared to an RNA oligonucleotide.

Some microRNA mimics, particularly those with extensive backbone and/or sugar modifications, are highly stable, showing little loss of activity at room temperatures for incubations as long as 1 year, and so may be particularly suitable for use in the present invention.

The miR-29 mimic or precursor may comprise one or more nucleotides comprising a modified sugar residue, i.e. a sugar residue other than a ribose residue. Examples of such modified sugar residues include 2′-O-methyl ribose, 2′-O-methoxyethyl ribose, 2′-fluoro-ribose and 4-thio-ribose, as well as bicyclic sugars. Bicyclic sugars typically comprise a furanosyl ring with a 2′,4′ bridge (e.g. a methylene bridge) which constrains the ring to the C3′ endo configuration. A nucleotide containing a bicyclic sugar is often referred to as a locked nucleic acid (“LNA”) residue.

The miR-29 mimic or precursor may independently contain one or more of any or all of these types of modified sugar residues. For example, the mimic may contain one, two, three, four, five, up to 10, up to 15, up to 20 or even more modified sugar residues. In certain embodiments, all nucleotides comprise a modified sugar residue.

Additionally or alternatively, the miR-29 mimic or precursor may comprise one or more backbone modifications, e.g. a modified internucleoside linkage.

Thus, one or more adjacent nucleotides may be joined via an alternative linkage moiety instead of a phosphate moiety.

It may be particularly desirable for a modified internucleoside linkage to be present at one or both ends of the miR-29 mimic, i.e. between the 5′ terminal nucleotide and the adjacent nucleotide, and/or between the 3′ terminal nucleotide and the adjacent nucleotide.

Moieties suitable for use as internucleoside linkages include phosphorothioate, morpholino and phosphonocarboxylate moieties, as well as siloxane, sulphide, sulphoxide, sulphone, acetyl, formacetyl, thioformacetyl, methylene formacetyl, thioformacetyl, alkenyl, sulphamate, methyleneimino, methylenehydrazino, sulphonate and sulphonamide moieties.

In a phosphorothioate moiety, a non-bridging oxygen atom is replaced by a sulphur atom. Phosphorothioate groups may promote serum protein binding and may thus improve in vivo distribution and bioavailability of the mimic. This may be desirable if the mimic is to be administered systemically to the recipient.

Additionally or alternatively, the miR-29 mimic or precursor may comprise one or more modified bases as alternatives to the naturally occurring adenine, cytosine, guanine and uracil. Such modified bases include 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

It has been suggested that the more heavily modified a passenger strand is, the less likely it is to be incorporated into the RISC complex, and thus the more effective the guide strand will be. Thus, even if the guide strand is a native miR-29, it may be desirable that the passenger strand comprises one or more modifications, e.g. one or more modified sugar residues, one or more modified inter-nucleoside linkages, and/or one or more modified bases.

Additionally or alternatively, a miR-29 mimic or precursor may comprise a membrane transit moiety, to facilitate transit across the target cell's plasma membrane. This moiety may be a suitable lipid or other fatty moiety, including but not limited to cholesterol and stearoyl moieties.

Other membrane transit moieties include cell penetrating peptides (“CPPs”, such as TAT and MPG from HIV-1, penetratin, polyarginine) and fusogenic peptides (e.g. endodomain derivatives of HIV-1 envelope (HGP) or influenza fusogenic peptide (diINF-7)). The membrane transit moiety may be conjugated to a carrier molecule which is non-covalently associated with the miR-29 mimic or precursor itself. Alternatively a membrane transit moiety may be conjugated to the miR-29 mimic or precursor itself.

The membrane transit moiety may be conjugated to either the guide strand or the passenger strand, although the passenger strand is preferred, so as not to impair guide strand function. Conjugation at either the 5′ or the 3′ terminus may be preferred, although conjugation to an internal residue is also possible.

For the avoidance of doubt, a miR-29 molecule (i.e. not otherwise possessing any structural or sequence differences from the native molecule) could be considered a miR-29 mimic or precursor when linked to a membrane transit moiety.

An example of a miR-29 mimic is the guide strand:

5′-rUrArGrCrArCrCrArUrCrUrGrArArArUrCrGrGmUmUmA-3′

where “r” indicates a ribose sugar and “m” indicates 2′-O-methyl ribose.

The guide strand may be part of a double stranded miR-29 mimic in combination with a passenger strand. Examples of suitable passenger strands are:

5′ mAmCrCmGrAmUrUmUrCmArGmArUmGrGmUrGmCrUA-3′
and
5′-mAmCrCmGrAmUrUmUrCmArGmArUmGrGmUrGmCrUmAdG-3′

Carriers for miR-29, Mimics and Precursors

The modulator may be provided in association with (e.g. complexed with or encapsulated by) a suitable carrier. Suitable carriers include pharmaceutically acceptable lipids and polymers, and combinations thereof. For example, the composition may have the form of liposomes, lipid vesicles, lipid complexes, polymer complexes or microspheres.

For example, lipid vesicles and liposomes are lipid bilayer particles having an aqueous core containing the oligonucleotide cargo.

Lipid complexes (or “lipoplexes”) and polymer complexes (“polyplexes”) typically contain positively charged lipids or polymers which interact with the negatively charged oligonucleotides to form complexes.

The cationic polymers or lipids may also interact with negatively charged molecules at the surface of the target cells. By suitable choice of lipids and head groups, the complexes can be tailored to facilitate fusion with the plasma membrane of the target cell or with a selected internal membrane (such as the endosomal membrane or nuclear membrane) to facilitate delivery of the oligonucleotide cargo to the appropriate sub-cellular compartment. Gene delivery by lipoplexes and polyplexes is reviewed, for example, by Tros de Ilarduya et al. in Eur. J. Pharm. Sci. 40 (2010) 159-170.

Neutral lipid emulsions may also be used to form particulate complexes with miRNAs having diameters of the order of nanometers.

Appropriate lipids may be selected by the skilled person depending on the application, cargo and the target cell. Single lipids may be used, or, more commonly, combinations of lipids.

Suitable lipids are described, for example, in WO2011/088309 and references cited therein, and include:
neutral lipids and phospholipids, such as sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, dilinoleoylphosphatidylcholine, phosphatidylcholine (PC), 1,2-Dioleoyl-sn-glycero-3-phosphocholine (DOPC), lecithin, phosphatidylethanolamine (PE), lysolecithin, lysophosphatidylethanolamine, sphinogomyelin (SM), cardiolipin, phosphosphatidic acid, 1,2-Distearoyl-sn-glycero-3-phosphocholine (DSPC), 1,2-Dipalmitoyl-sn-glycero-3-phosphoethanolamine (DPPE), 1-Palmitoyl-2-oleoyl-sn-glycero-3-phosphocholine (POPC), 1,2-Dilauroyl-sn-glycero-3-phosphocholine (DLPC), 1,2-Dimyristoyl-sn-glycero-3-phosphocholine (DMPC), 1,2-Dipalmitoyl-sn-glycero-3-phosphocholine (DPPC), 1,2-Dimyristoyl-sn-glycero-3-phosphoethanolamine (DMPE), 1,2-Dioleoyl-sn-glycero-3-phosphoethanolamine (DOPE), dipalmitoloeoyl-PE, diphytanoyl-PE, DSPE, dielaidoyl-PE, dilinoleoyl-SM, and dilinoleoyl-PE;
sterols, e.g. cholesterol
polymer-modified lipids, e.g. polyethylene glycol (PEG) modified lipids, including PEG-modified phosphatidylethanolamine and phosphatidic acid, PEG-ceramide conjugates, PEG-modified dialkylamines and PEG-modified 1,2-diacyloxypropan-3-amines. Particularly suitable are PEG-modified diacylglycerols and dialkylglycerols, e.g. PEG-didimyristoyl glycerol (PEG-DMG) PEG-distyryl glycerol (PEG-DSG) and PEG-carbamoyl-1,2-dimyristyloxypropylamine (PEG-cDMA);
cationic lipids, such as N,N-dioleyl-N,N-dimethylammonium chloride (“DODAC”); N-(2,3-dioleyloxy)propyl-N,N—N-triethylammonium chloride (“DOTMA”); N,N-distearyl-N,N-dimethylammoniumbromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3β-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), N-(1,2-dimyristyloxyprop-3-yl)-N,N-dimethyl-N-hydroxyethyl ammonium bromide (“DMRIE”), 1,2-dilinoleyloxy-3-dimethylaminopropane (DLinDMA) 1,2-Dilinoleoyl-3-dimethylaminopropane (DLinDAP), 1-Linoleoyl-2-linoeyloxy-3-dimethylaminopropane (DLin-2-DMAP), 1,2-Dilinoleylcarbamoyloxy-3-dimethylaminopropane (DLin-C-DAP), 1,2-Dilinoleylthio-3-dimethylaminopropane (DLin-S-DMA), and 2,2-Dilinoleyl-4-10 dimethylaminomethyl-[1,3]-dioxolane (DLin-K-DMA). Commercial preparations of cationic lipids include Lipofectin™ (comprising DOTMA and DOPE, available from Gibco/BRL), and Lipofectamine™ (comprising DOSPA and DOPE, available from Gibco/BRL).
anionic lipids including phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine and lysylphosphatidylglycerol.

W0/0071096 describes different formulations, such as a DOTAP:cholesterol or cholesterol derivative formulation that can effectively be used for oligonucleotide delivery.

A commercially available composition capable of achieving good delivery of miRNA to tissues is the neutral lipid emulsion MaxSuppressor in vivo RNALancerII (BIOO Scientific, Austin, Tex.) which consists of 1,2-dioleoyl-sn-glycero-3-phosphocholine, squalene oil, polysorbate 20 and an antioxidant. In complex with synthetic miRNAs, it forms nanoparticles in the nanometer diameter range.

Suitable polymers include histones and protamines (and other DNA-binding proteins), poly(ethyleneimine) (PEI), cationic dendrimers such as polyamidoamine (PAMAM) dendrimers, 2-dimethyl(aminoethyl) methacrylate (pDMAEM), poly(L-lysine) (PLL), carbohydrate-based polymers such as chitosan, etc. See Tros de Ilarduya et al. in Eur. J. Pharm. Sci. 40 (2010) 159-17 for a review.

Microsphere drug delivery systems have been fabricated from biodegradable polymers by a variety of techniques including combinations of phase separation or precipitation emulsion/solvent evaporation and/or spraying methods. Microspheres are typically between 1-100 μm in diameter. Any suitable polymer, such as those described above, may be used. Drugs may be incorporated into the particles in several different ways depending on the properties of the drug. Hydrophobic therapeutics may be co-dissolved with the polymer in a solvent such as methylene chloride or ethyl acetate. Hydrophilic therapeutics, including proteins, may be suspended in the organic phase as a finely ground dry powder. Alternatively, an aqueous solution of a hydrophilic therapeutic may be mixed with an organic polymer solution to form a water-in-oil emulsion. See Varde, N K and Pack, D W, Expert Opin. Biol. Ther. (2004) 4(1), 35-51 for a review.

Proteins and peptides such as atellocollagen can also be used. Atellocollagen is a water soluble form of collagen produced by protease treatment, in particular pepsin-treated type I collagen from calf dermis.

Cyclodextrins may also be of use for delivery.

### Targeting Agents

Carrier molecules may also carry targeting agents capable of binding to the surface of the target cell. For example, the targeting agent may be a specific binding partner, capable of binding specifically to a molecule expressed on the surface of a target tendon cell. Suitable binding partners include antibodies and the like, directed against cell surface molecules, or ligands or receptors for such cell surface molecules. Surface markers which may assist in targeting to tendon cells include Tenascin C, CD55 and tenomodulin.

The term “specific binding pair” is used to describe a pair of molecules comprising a specific binding member (sbm) and a binding partner (bp) therefor which have particular specificity for each other and which in normal conditions bind to each other in preference to binding to other molecules. Examples of specific binding pairs are antibodies and their cognate epitopes/antigens, ligands (such as hormones, etc.) and receptors, avidin/streptavidin and biotin, lectins and carbohydrates, and complementary nucleotide sequences.

It is well known that fragments of a whole antibody can perform the function of binding antigens. Examples of functional binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P. Holliger et al Proc. Natl. Acad. Sci. USA 90 6444-6448, 1993).

As antibodies can be modified in a number of ways, the term “antibody” should therefore be construed as covering any specific binding substance having an binding domain with the required specificity. Thus, this term covers the antibody fragments described above, as well as derivatives, functional equivalents and homologues of antibodies, including any polypeptide comprising an immunoglobulin binding domain, whether natural or synthetic. Chimaeric molecules comprising an immunoglobulin binding domain, or equivalent, fused to another polypeptide are therefore included. Cloning and expression of chimaeric antibodies are described in EP-A-0120694 and EP-A-0125023.

Alternatives to antibodies are increasingly available. So-called “affinity proteins” or “engineered protein scaffolds” can routinely be tailored for affinity against a particular target. They are typically based on a non-immunoglobulin scaffold protein with a conformationally stable or rigid core, which has been modified to have affinity for the target. Modification may include replacement of one or more surface residues, and/or insertion of one or more residues at the surface of the scaffold protein. For example, a peptide with affinity for the target may be inserted into a surface loop of the scaffold protein or may replace part or all of a surface loop of the scaffold protein. Suitable scaffolds and their engineered equivalents include:
BPTI, LAC-DI, ITI-D2 (Kunitz domain scaffolds);
ETI-II, AGRP (Knottin);
thioredoxin (peptide aptamer);
Fn3 (AdNectin);
lipocalin (BBP) (Anticalin);
ankyrin repeat (DARPin);
Z domain of protein A (Affibody);
gamma-B-crystallin/ubiquitin (Affilin);
LDLR-A-domain (Avimer).

See, for example, Gebauer, M and Skerra, A, Current Op. Chem. Biol. 2009, 13: 245-255, and Friedman, M and Stahl, S, Biotechnol. Appl. Biochem. (2009) 53: 1-29, and references cited therein.

Nucleic Acids Encoding miR-29, Mimics and Precursors

As an alternative to miR-29 oligonucleotides, mimics and precursors, intended to be taken up directly by a target cell, it is possible to employ a nucleic acid encoding a miR-29 oligonucleotide, a mimic thereof, or a precursor of either, to be taken up by the target cell such that the miR-29 oligonucleotide, mimic or precursor is expressed within the target cell. Such an approach may be regarded as “gene therapy”.

It will be readily apparent to the skilled person that nucleic acids can only be used to encode miR-29, mimics and precursors thereof composed of RNA, i.e. composed of the four naturally occurring nucleotide components of RNA, without modified bases, sugars or internucleoside linkages.

The nucleic acid typically comprises an expression construct, comprising a nucleic acid sequence encoding the miR-29 oligonucleotide, mimic or precursor, operably linked with appropriate regulatory sequences to facilitate expression. The regulatory sequences may be selected depending on the target cell, but will typically include an appropriate promoter and optionally one or more enhancer sequences which direct transcription by RNA polymerase II, as well as a transcriptional terminator (normally including a polyadenylation signal).

The promoter may be a tissue-specific promoter, which drives transcription preferentially or exclusively in the target cell or tissue as compared to other cell or tissue types.

Thus, the promoter may be a promoter which drives transcription preferentially or exclusively in tendon cells. The collagen 1a1 (col1a1) promoter may be a suitable promoter.

The expression construct may form part of an expression vector. The skilled person will be capable of designing suitable nucleic acid expression constructs and vectors for therapeutic use. The vectors will typically contain an expression construct as described above, optionally combined with other elements such as marker genes and other sequences depending upon the particular application. The vectors may be intended to integrate into a host cell chromosome, or may exist and replicate independently of the host chromosomes as an episome, e.g. a plasmid.

The nucleic acid may be employed in naked form, associated with (e.g. complexed with or encapsulated by) a suitable carrier such as a polymer or lipid (as described elsewhere in this specification), or coated onto a particulate surface. In such embodiments, the nucleic acid is typically DNA. The nucleic acid or carrier may also comprise a targeting moiety or membrane transport moiety as described elsewhere in this specification in relation to miR96, precursors and mimics themselves.

Alternatively, the nucleic acid may be provided as part of a viral vector.

Any suitable type of viral vector may be employed as a gene delivery vehicle. These include adenovirus, adeno-associated virus (AAV), retrovirus (especially lentivirus) and herpesvirus vectors. Adenovirus and lentivirus may be particularly preferred as they have the capacity to achieve expression of the gene(s) delivered in cells which are not actively dividing.

The viral vector typically comprises viral structural proteins and a nucleic acid payload which comprises the desired expression construct in a form functional to express the gene in the target cell or tissue. Thus the gene is typically operably linked to a promoter and other appropriate transcriptional regulatory signals.

In adenoviral vectors, the nucleic acid payload is typically a double stranded DNA (dsDNA) molecule. In retroviral vectors, it is typically single stranded RNA.

The nucleic acid payload typically contains further elements required for it to be packaged into the gene delivery vehicle and appropriately processed in the target cell or tissue.

For adenoviral vectors, these may include adenoviral inverted terminal repeat (ITR) sequences and an appropriate packaging signal.

For retroviral vectors, these include characteristic terminal sequences (so-called “R-U5” and “U3-R” sequences) and a packaging signal. The terminal sequences enable the generation of direct repeat sequences (“long terminal repeats” or “LTRs”) at either end of the provirus which results from reverse transcription, which then facilitate integration of the provirus into the host cell genome and direct subsequent expression.

The nucleic acid payload may also contain a selectable marker, i.e. a gene encoding a product which allows ready detection of transduced cells. Examples include genes for fluorescent proteins (e.g. GFP), enzymes which produce a visible reaction product (e.g. beta-galactosidase, luciferase) and antibiotic resistance genes.

The viral vector is typically not replication-competent. That is to say, the nucleic acid payload does not contain all of the viral genes (and other genetic elements) necessary for viral replication. The viral vector will nevertheless contain all of the structural proteins and enzyme activities required for introduction of the payload into the host cell and for appropriate processing of the payload such that the encoded miR-29, mimic or precursor can be expressed. Where these are not encoded by the nucleic acid payload, they will typically be supplied by a packaging cell line. The skilled person will be well aware of suitable cell lines which can be used to generate appropriate viral delivery vehicles.

Thus, for an adenoviral vector, the nucleic acid payload typically lacks one or more functional adenoviral genes from the E1, E2, E3 or E4 regions. These genes may be deleted or otherwise inactivated, e.g. by insertion of a transcription unit comprising the heterologous gene or a selective marker.

In some embodiments, the nucleic acid contains no functional viral genes. Thus, for an adenoviral vector, the only viral components present may be the ITRs and packaging signal.

Nucleic acids having no functional viral genes may be preferred, as they reduce the risk of a host immune response developing against the transduced target cell or tissue as a result of viral protein synthesis.

Viral vectors may be engineered so that they possess modified surface proteins capable of binding to markers on the target cell, thus increasing the chance that the desired target cell will be transduced and reducing the chance of non-specific transduction of other cell or tissue types. This approach is sometimes referred to as pseudotyping. Thus the viral vector may comprise a surface protein capable of binding to a surface marker on a tendon cell. Surface markers which may assist in targeting to tendon cells include Tenascin C and CD55.

### The Tendon and Tendon Damage

Tendons are the connective tissue attaching muscle to bone. They allow the transduction of force from a contracting muscle to be exerted upon the attached skeletal structure at a distance from the muscle itself′.

Tendons are a complex, systematically organised tissue and comprise several distinct layers.

The tendon itself is a roughly uniaxial composite comprising around 30% collagen and 2% elastin (wet weight) embedded in an extracellular matrix containing various types of cells, most notably tenocytes³.

The predominant collagen is type I collagen, which has a large diameter (40-60 nm) and links together to form tight fibre bundles. Type 3 collagen is also present and is smaller in diameter (10-20 nm), forming looser reticular bundles.

The collagen is organised (in increasing complexity) into fibrils, fibres, fibre bundles and fascicles, surrounded by a layer of loose, collagenous and lipid-rich connective tissue matrix known as the endotenon⁴. A layer of the same material, called the epitenon, covers the surface of the entire tendon. Surrounding the epitenon is a connective tissue called the paratenon which contains type 1 and type 3 collagen fibrils, some elastic fibrils and a layer of synovial cells. Some tendons are additionally surrounded by a tendon sheath.

The major cell types within the tendon are tenocytes and tenoblasts, both of which are fibroblast-like cells¹⁴. Both types of cells are important in the maintenance of healthy tendon, as both produce collagen and maintain the extracellular matrix¹⁵. Thus the term “tendon cell” as used in this specification encompasses both tenocytes and tenoblasts.

Tenocytes are flat, tapered cells, spindle shaped longitudinally, and stellate in cross section, and are detected sparingly in rows between collagen fibres. They have elaborate cell processes forming a three dimensional network extending through the extracellular matrix, communicate via cell processes, and may be motile.

Tenoblasts are precursors of tenocytes. They are spindle shaped or stellate cells with long, tapering, eosinophilic flat nuclei. They are motile and highly proliferative.

During embryonic development, tenoblasts and hence tenocytes originate from mesodermal compartments, as do skeletal myoblasts, chondrocytes and osteoblasts¹⁶. Some of the multipotent mesenchymal progenitor cells that arise from these compartments express the basic helix-loop-helix transcription factor scleraxis. However, once they are committed to become cells making up a specific tissue, only tenoblasts and tenocytes retain the ability to express scleraxis. The scleraxis gene is thus the first master gene found to be essential for establishing the tendon lineage during development. Tenomodulin is a type II transmembrane glycoprotein induced in mouse tendons in a late (embryonic day [E] 17.5) developmental phase and is also observed in adult tendons. Thus scleraxis represents a marker for both tenoblasts and tenocytes, while tenomodulin is a surface marker for mature tenocytes¹⁹.

Tendon injury or damage may be caused by or associated with numerous factors including (but not limited to) external trauma, mechanical stress (including over-use), degeneration, inflammation, and combinations of these, often referred to as “tendinopathy”. It may include tendon rupture (i.e. complete failure of the tendon).

Tendinopathy is multifactorial, has a spectrum from acute to chronic, and is often associated with over-use of the tendon, which may be instantaneous or over an extended period of time. Tendinopathy may involve degeneration or other kinds of mechanical damage to the collagen at a microscopic or macroscopic level (sometimes referred to as “tendinosis”), inflammation, or a combination of both (sometimes referred to as “tendinitis”).

The biomechanical properties of tendon, especially its tensile strength, are related to cross-sectional area (i.e. thickness), collagen content, and the ratio between different types of collagen. After acute injury, during tendinopathy, and during healing of tendon damage, a shift occurs in collagen synthesis, away from type 1 collagen toward type 3 collagen. Type 1 collagen synthesis may return to normal levels after an initial drop, but a persistent increase in type 3 synthesis leads to a long-term imbalance in collagen ratio. This has a significant and deleterious effect on the biomechanical properties of the tendon. In particular, it reduces the tensile strength of the tendon, reducing its ultimate failure strength and thus making it more prone to subsequent rupture.

The implants of the invention are typically employed as part of a surgical procedure to repair, or facilitate healing of, tendon damage or injury. This includes injury resulting from the surgical procedure itself.

The implants of the invention may be applied to any damaged tendon. The main tendons affected by tendinopathy in humans are the Achilles tendon, the supraspinatus tendon, the common flexor tendon and the common extensor tendon. The main tendon affected by tendinopathy in equine subjects is the superficial flexor tendon. These may represent particularly significant targets for treatment.

### Tendon Scaffolds

Tissue engineering techniques using biocompatible materials offer various options for managing tendon disorders and healing^{46,47,48}. Preliminary studies support the idea that exogenous implants such as scaffolds have significant potential for tendon augmentation with an enormous therapeutic potential⁴⁹, although definitive conclusions are not yet possible.

The term “scaffold” is typically used to describe an artificial structure which is used to support formation of three-dimensional biological tissue. Thus, in the context of the present invention, the substrate can be regarded as a scaffold.

In use, a scaffold may be located along or around a tendon, so that it extends over or across a lesion in need of repair. For such uses, the substrate may be a web or sheet of appropriate material, to be formed around the tendon to which it is applied.

Alternatively, a scaffold may be used as a replacement for part or all of a tendon. Thus it may be used to replace a tendon in its entirety or it may form an insert into a tendon, e.g. between two portions of native tendon or at the interface between a portion of native tendon and bone (i.e. at the enthesis). In such embodiments, the substrate will provide a three-dimensional template to guide the growth of regenerating tendon tissue. The substrate may therefore have a cord-like or rod-like configuration, with a cross-section mimicking that of native tendon.

Whatever the form or configuration of the scaffold, the substrate is capable of supporting growth of tendon cells. By this is meant that tendon cells are capable of adhering to it and performing their normal biological functions, which may include metabolism, migration, replication and generation of ECM depending on the cell type in question.

It is normally desirable that the substrate is composed of bioresorbable materials, to reduce or eliminate the need for removal.

The substrate may be absorbed into the structure of the tendon as cells grow around and through it.

The substrate is typically porous to allow such cell growth. For example, it may comprise a fabric of woven or unwoven fibres. Alternatively the substrate may comprise a matrix or foam. For example, the substrate may comprise a gel, such as a hydrogel.

The mean pore diameter may be in the range of 10-500 μm, e.g. 50-500 μm, e.g. 100-500 μm or 200-500 μm. For optimum growth of soft tissue, it has been proposed that a minimum mean pore diameter of 200 μm may be desirable.

The substrate may comprise or consist of extra-cellular matrix (ECM).

The ECM may be derived from a tissue explant, e.g. from connective tissue (such as tendon), small intestinal submucosa (SIS), dermis or pericardium. The explant may be derived from any suitable species or source. The source will typically be mammalian, e.g. human, porcine, bovine or equine. The explant may be derived from the same species as the intended recipient, although this may not always be practicable.

ECM may also be laid down by a suitable cell population or tissue in culture (e.g. in vitro or ex vivo) for use as a scaffold substrate.

Whatever the source of the ECM, it may be desirable to remove cellular material and other non-ECM components (such as lipids and fat deposits). This may help to reduce the risk of host rejection while retaining the natural ECM structure. Thus preparation of the ECM may involve a step of decellularisation (e.g. comprising treatment with an appropriate protease such as trypsin), oxidation (e.g. with peracetic acid), freeze drying, or any combination thereof. Additionally or alternatively the ECM may be chemically cross-linked to increase or maintain its natural mechanical properties.

The final substrates prepared by such techniques are typically composed mainly of collagen fibres, predominantly type I collagen, and may have a surface chemistry and native structure that is bioactive and capable of promoting cellular proliferation and tissue in growth⁴⁶.

The resulting ECM may be sterilized prior to use.

Where porcine tissues are used as the basis for scaffold materials, especially for use in a different species (such as humans), they may be obtained from alpha-1,3-galactosyl transferase-deficient porcine tissue. This may help to minimise any immune response against the porcine tissue when implanted into the recipient species.

Alternatively, the substrate may be a synthetic substrate, e.g. a substrate formed other than by biological cells. A synthetic substrate may nevertheless comprise biological components (i.e. components which occur in nature) such as proteins, polysaccharides and other biological polymers, as well as synthetic components (i.e. components which do not occur in nature) such as synthetic polymers.

Suitable proteins include collagen, elastin, fibrin, albumin and gelatin. Suitable polysaccharides include hyaluronan (also known as hyaluronic acid and hyaluronate) alginate (also known as alginin or alginic acid) and chitosan. Many of these, such as collagen, elastin and hyaluronan are natural components of the extracellular matrix.

Suitable synthetic components include biocompatible synthetic polymers. The skilled person is well aware of many suitable such polymers including polyvinyl alcohol, oligo[poly(ethylene glycol) fumarate] (OPF), and polymers and co-polymers of monomers such as glycolic acid and lactic acid, such as poly(glycolic acid) (PGA), poly(lactic acid) (PLA) and poly(lactic-co-glycolic acid) (PLGA). The monomers may be in the D or L form, or mixture of both, as desired. The skilled person will be capable of determining appropriate ratios of the respective monomers depending on the desired properties of the implant.

Suitable cross-linking agents may be employed as necessary, e.g. in formation of a matrix. Suitable cross linking agents are well known to the skilled person. For example, OPF-based hydrogels have been cross-linked using poly(ethylene glycol) diacrylate (PEG diacrylate) and poly(ethylene glycol) dithiol (PEG-dithiol).

A gel is commonly recognised to be a substance with properties intermediate between the solid and liquid states. Gels are essentially colloidal, with a disperse solid phase and a continuous liquid phase. The solid phase is typically an extended three-dimensional network or matrix, often of polymeric material, which may be cross-linked. The liquid phase is commonly water (or an aqueous solution) and such gels are often referred to as hydrogels. Hydrogels are particularly suitable for use in the present invention. The hydrogel may be a thermosensitive sol-gel transition hydrogel. Thus a gel may also be seen as a form of matrix-containing substrate.

The matrix components described above may all be suitable for use as the matrix component of a gel substrate. Thus, a gel may, for example, comprise alginate, hyaluronan, collagen, gelatin, fibrin, albumin, polymers or copolymers of glycolic acid and lactic acid, etc. A platelet-rich plasma (PRP) gel may also be suitable.

Additionally or alternatively, the substrate may comprise or consist of a bioceramic material, such as hydroxyl carbonate apatite (HCA) or tricalcium phosphate, or a biodegradable metallic material, such as porous magnesium or magnesium oxide.

The substrate may be composed of a plurality of layers, for example it may comprise a plurality of layers of fabric or ECM. The substrate may comprise a gradient structure, mimicking the transition from collagen to bone at the enthesis. The gradient may represent increasing hardness and/or increasing mineralisation (calcification), e.g. as described in references 47 and 48.

Even when the substrate is not principally composed of extracellular matrix, it may nevertheless be desirable that the substrate comprises some proportion of one or more extracellular matrix components, such as collagen, elastin, hyaluronan, etc. Their presence may assist cell adhesion, replication and migration on and through the substrate. If desired, a substrate may be coated with one or more extracellular matrix components.

The substrate may further comprise one or more modulators of cell adhesion or cell growth. For example, cell adhesion peptides may be incorporated to promote cell adhesion. Such peptides may comprise or consist of integrin binding motifs such the tripeptide Arg-Gly-Asp (RGD) and the tetrapeptide Arg-Gly-Asp-Ser (RGDS) as well as heparin binding peptides. Whatever the composition of the substrate, cell adhesion (as well as replication and migration) may also be assisted by the presence of growth factors on or within the substrate. Such growth factors may include bFGF (basic fibroblast growth factor, also designated FGF2 or FGF-beta) and TGF-beta (transforming growth factor beta) and PDGF (Platelet derived growth factor),

Modulators of cell adhesion or cell growth such as cell adhesion peptides, growth factors, etc. may be adsorbed onto the surface of the substrate (e.g. via non-covalent interactions such as hydrogen bonding or hydrophobic interactions) or may be covalently coupled to the surface (e.g. via a linker molecule or tether). Flexible tethers for attaching growth effector molecules to a substrate should satisfy (1) the need for mobility of the ligand-receptor complex within the cell membrane in order for the effector molecule to exert an effect, and (2) the need for biocompatibility. Substantial mobility of a tethered growth factor is important because, even though the cell does not need to internalize the complex formed between the receptor and the growth factor, it is believed that several complexes must cluster together on the surface of the cell in order for the growth factor to stimulate cell growth. In order to allow this clustering to occur, the growth factors are attached to the solid surface, for example, via long water-soluble polymer chains, allowing movement of the receptor-ligand complex in the cell membrane.

Examples of water-soluble, biocompatible polymers which can serve as tethers include polymers such as polyethylene oxide (PEO), polyvinyl alcohol, polyhydroxyethyl methacrylate, polyacrylamide, and natural polymers such as hyaluronic acid, chondroitin sulfate, carboxymethylcellulose, and starch.

It will be understood that reference in this context to the “surface” of the substrate encompasses the internal surfaces of any matrix or foam from which the substrate is composed.

Where the substrate is a gel, the cell adhesion peptides and/or growth factors may be suspended or dissolved in the liquid phase.

The substrate may comprise one or more cells. Suitable cells may include tendon cells such as tenocytes or tenoblasts, and precursors thereof such as mesenchymal stem cells. One or more cells may be applied to the substrate prior to introduction of the substrate at the target site. Alternatively, one or more cells may be applied to the substrate after introduction of the substrate. Such application of cells to the substrate is often referred to as “seeding” the substrate with cells.

Thus the invention extends to a method of preparing an implant of the invention comprising providing a substrate as described herein, contacting said substrate with a tendon cell or a precursor thereof, and culturing the substrate. Such methods enable the production of a cellularised or partially cellularised implant in vitro or ex vivo and may assist in the formation of appropriate ECM before introduction of the implant to the recipient.

The implant of the invention further comprises a modulator of tendon healing, which is

(i) miR-29, a mimic thereof, or a precursor of either; or
(ii) a nucleic acid encoding miR-29, a mimic thereof, or a precursor of either.

The modulator may be attached to or incorporated into the substrate before introduction of the implant at the target site.

Thus, the substrate may be impregnated with the modulator before introduction to the target site.

The modulator may be admixed with components of the substrate prior to formation of the substrate. This may be particularly appropriate for gel substrates, where the modulator may be admixed with one or more components of the gel prior to gelation.

Alternatively impregnation may occur after formation of the substrate, e.g. by immersion of the substrate in a solution of the modulator. The modulator may be provided in an aqueous solution, e.g. physiologically compatible or pharmaceutically acceptable solution, such as physiological saline solution or PBS. Immersion may be for any suitable period of time to allow adequate absorption of the modulator by the substrate, or adsorption onto the surface of the substrate as the case may be. Typically, periods of between 5 minutes and 48 hours are normally adequate, e.g. 1 hour to 48 hours, e.g. 12 hours to 48 hours, e.g. 24 hours to 48 hours.

Immersion (or “dip-coating” may be particularly suitable for polymer and ECM substrates.

The same technique may be used to apply modulators of cell adhesion or cell growth (such as cell adhesion peptides, growth factors etc. as described above) to the substrate.

Alternatively, the substrate may be introduced at the target site and the modulator subsequently applied to the substrate, e.g. by coating onto the substrate surface or by injection into the substrate.

A gel substrate may be formed or set in situ at the target site. In such embodiments, the modulator may be admixed with one or more components of the gel prior to gelation, or may be applied to the gel after gelation.

Therapeutic Application of miR-29, Mimics and Precursors

The inventors have found that, by increasing miR-29 activity in tendon cells, it is possible to alter the collagen balance in favour of type 1 collagen synthesis and away from type 3 collagen synthesis.

Thus, the invention provides methods for modulating the healing of tendon by therapeutic application of miR-29. The methods described in this specification may be regarded as methods for modulating relative collagen composition and/or synthesis in the tendon, in particular the relative content and synthesis of type 1 and type 3 collagen in the tendon. The balance is believed to be modulated in favour of type 1 collagen, i.e. increasing collagen 1 synthesis or content within the tendon relative to type 3 collagen. It will be appreciated that this does not necessarily involve a net increase in type 1 collagen synthesis or content, as miR-29 may inhibit type 1 collagen synthesis. However, synthesis of type 3 collagen is inhibited to a greater extent than that of type 1 collagen.

At a physiological level, the methods described in this specification may be regarded as methods for modulating the biomechanical properties of the tendon, preferably improving the biomechanical properties of the tendon, e.g. improving or increasing the tensile strength of the tendon.

The methods of the invention may be applied at any stage of tendinopathy, or at any stage of the healing process of an injured tendon. For example, the methods may be used to modulate the collagen ratio, and hence the biomechanical properties of the tendon, during healing of tendinopathy or during healing of an acute tendon injury such as a ruptured tendon.

Thus the methods of the invention may equally be regarded as methods for the treatment of tendon damage, including damage resulting from tendon injury and tendinopathy.

IL-33 may be observed in tendon for a short period after injury and in the early stages of tendinopathy. Without wishing to be bound by any particular theory, IL-33 may be implicated in the switch from type 1 to type 3 collagen synthesis. However, the imbalance in collagen synthesis is believed to persist after the initial involvement of IL-33.

The methods of the invention are not restricted to treatment in the early stages of tendon injury, but are equally applicable to later stage injury or disease, e.g. chronic tendinopathy.

Thus treatment may be administered at any stage after onset of symptoms or after a traumatic event causing damage to the tendon. For example, treatment may be administered 1 day, 2 days, 3, days, 4, days, 5 days, 6 days, 7 days or more after onset of symptoms or a traumatic event. It may be administered, 1 week, 2 weeks, 3 weeks, 4 weeks or more after onset of symptoms or a traumatic event. It may be administered 1 month, 2 months, 3 months, 4 months, 5 months, 6 months or more after onset of symptoms or a traumatic event.

### Subjects for Treatment

Although the most common subjects for treatment will be humans, the methods of the invention may extend to any other mammals, including other primates (especially great apes such as gorilla, chimpanzee and orang utan, but also Old World and New World monkeys) as well as rodents (including mice and rats), and other common laboratory, domestic and agricultural animals (including but not limited to rabbits, dogs, cats, horses, cows, sheep, goats, etc.).

The methods may be particularly applicable to equine subjects, i.e. horses. Horses, and especially thoroughbred horses such as racehorses, are particularly prone to tendon injuries. Given the value of many of the animals concerned, there is a long-standing need for effective treatments.

### Compositions for Application of Modulators

Compositions for use in the present invention (e.g. compositions comprising modulators for administration to a substrate) will conventionally be formulated as pharmaceutically acceptable compositions. These compositions may comprise, in addition to the modulator itself, a pharmaceutically acceptable excipient, carrier, buffer, stabiliser or other materials well known to those skilled in the art. Such materials should be non-toxic and should not interfere with the efficacy of the active ingredient.

Since the modulator will typically be applied at the site of injury, the composition may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors and veterinary practitioners, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

The invention will now be described in more detail, by way of example and not limitation, by reference to the accompanying drawings and examples.

### DESCRIPTION OF THE DRAWINGS

FIG. 1: IL-33/ST2 expression in tendon.

(A) IL-33, (B) soluble ST2 (sST2) and (C) membrane ST2 (mST2) gene expression in tendon samples. Fold change in gene expression of IL-33, Soluble/Membrane ST2 in control (n=10), torn supraspinatus and matched subscapularis human tendon samples (n=17). Data points shown are relative expression compared to housekeeping gene 18S (mean of duplicate analysis). Mean±SD reflects patient population comparisons by t-test. (D) Modified Bonar scoring for samples of tendon with mean and SEM shown. n=10 for control tendon (Ctl), n=17 for torn tendon and early tendinopathy. Modified Bonar scoring system depicts mean score per sample based on 10 high power field. 0=no staining, 1=<10%, 2=10-20%, 3=>20%+ve staining of cells per high power field. (E) Fold change in gene expression of IL-33, and ST2, 24 hours post incubation with respective doses of TNFα alone, IL-1β alone and in combination. Data shown as the mean±SD of triplicate samples and are in turn, representative of experiments performed on three individual patient samples. *p<0.05, **p<0.01 compared to control samples. (F) Fold change in gene expression of coil and col3 with 50 and 100 ηg/ml rhIL-33 24 hours post incubation. (G) Time course for coil and col3 gene expression following incubation with 100 ηg/ml IL-33. (H) Collagen 1 and 3 protein expression 24 hours post incubation with increasing concentrations of rhIL-33. For F, G and H, data are shown as the mean±SD of triplicate samples and are in turn, representative of experiments performed on three individual patient samples. *p<0.05, **p<0.01 compared to control samples.

FIG. 2: IL-33/ST2 axis in tendon healing in vivo.

(A,B) IL-33 gene expression and soluble ST2 gene expression on Days 1,3,7 and 21 post injury. Data shown are the mean fold change±SD (pooled data from 4 mice per group performed on four sequential occasions therefore n=16 per condition) *p<0.05, **p<0.01 control versus injured mice. (C,D) coil mRNA and collagen 1 protein levels in WT and ST2−/− post injury on Days 1 and 3 post injury. (E,F) col3 mRNA and collagen 3 protein levels in WT and ST2−/− on days 1 and 3 post injury. Data shown are mean±SD of duplicate samples and are representative of experiments using four mice per condition (n=16). *p<0.05, **p<0.01 control versus injured mice. +p<0.05, ++p<0.01 WT injured versus ST2−/− injured mice. (G) percentage change in tendon strength for WT and ST2−/− injured and uninjured tendons on days 1 and 3 post injury. Data are shown as the mean±SD and are representative of experiments using four mice per condition (n=16). *p<0.05, **p<0.01 control versus injured mice. # p<0.05 ST2−/− injured versus WT injured mice.

FIG. 3: IL-33 promotes collagen 3 production and reduced tendon strength while anti IL-33 attenuates these changes in tendon damage in vivo.

(A) coil mRNA, (B) Collagen 1 protein, (C) col3 mRNA and (D) Collagen 3 protein in WT and ST2−/− mice treated with rhIL-33 on Day 1 post injury. Data are shown as the mean±SD of duplicate samples and are representative of experiments using four mice per condition (n=16). *p<0.05,**p<0.01, injured versus uninjured mice. +p<0.05 WT versus ST2−/− mice. (E) percentage change in tendon strength in WT uninjured mice on Days 1 and 3 post treatment with rhIL-33. Data are shown as the mean±SD and are representative of experiments using four mice per group (n=16). **p<0.01, injured versus uninjured mice. (F) coil mRNA, (G) collagen 1 protein, (H) col3 mRNA and (I) collagen 3 protein levels post treatment with anti-IL-33 at days 1 and 3 post tendon injury in WT mice. (J) percentage change in tendon strength in anti IL-33 treatment WT mice on days 1 and 3 post injury. Data are shown as the mean±SD and are representative of experiments using four mice per condition (n=16). *p<0.05,**p<0.01, injured versus uninjured mice. A-J, Data are shown as the mean±SD of duplicate samples and are representative of experiments using four mice per condition (n=16)

FIG. 4: MicroRNA 29 directly targets soluble ST2-implications for collagen matrix changes in tendon disease.

(A) All members of the miR-29 family (miR-29a, miR-29b, and miR-29c) were expressed in tendinopathic tenocytes (n=6 patient samples). Lower ΔCt values indicate higher levels of expression. miR-29 family gene expression in Control, torn supraspinatus (Torn Tendon) and matched subscapularis tendon (Early Tendinopathy). Data shown as the mean±SD of duplicate samples and represent experiments on ten patient samples. *p<0.05, **p<0.01. (B) Time course of miR-29a expression following the addition of 100 ng/ml of rhIL-33. (C&D) coil and col3 mRNA and Collagen 1 and 3 protein expression following transfection with scrambled mimic, miR-29a mimic or miR29a antagomir. (E) Collagen 3 protein levels following addition of miR-29a mimic/antagomir and 100 ng rhIL-33. For B-E data shown are the mean±SD of duplicate samples and represent experiments on five tendon explant samples. (n=5) p<0.05, **p<0.01 (F) Luciferase activity in primary human tenocytes transfected with precursor miR-29a containing 3′UTR of Col 1a1, Col1a2 or Col 3a1. Activity was determined relative to controls transfected with scrambled RNA, which was defined as 100%. This was repeated in 3 independent experiments. * p<0.05, **p<0.01 versus scrambled control. (G) miR-29a binding sites and MRE's on col3a1 and col1a1/col1a2 long/short forms highlighting alternative polyadenisation sites. (H) percentage of long/short collagen transcripts in tenocytes (T) following transfection with miR-29a. (I) col1a1, col1a2 and col3a1 mRNA following transfection with scrambled mimic and miR-29a antagomir. Data shown are the mean±SD of duplicate samples and represent experiments on three tendon explant samples. (n=3) p<0.05, **p<0.01

FIG. 5: IL-33/ST2 regulates miR-29 in tendon healing in vivo

(A) Cotransfection of HEK 293 cells with pre-miR-29a containing 3′UTR of soluble ST2 together with miRNA Regulatory Elements (MRE's) of 3′UTR of soluble ST2 and resultant luciferase activity assay. *** p<0.001 versus scrambled control (n=3) (B) sST2 and membrane bound ST2 mRNA levels following addition of scrambled mimic miR-29a mimic or miR-29a antagomir (C) human sST2 protein production (ng/ml) following incubation with miR29a mimic/antagomir. (n=5) p<0.05, **p<0.01.

(D) Quantitative PCR showing mean fold change±SD in miR-29a in WT injured versus uninjured animals on days 1 and 3 post injury. (E) Quantitative PCR showing mean fold change±SD in miR-29a in WT and ST2−/− mice in injured versus uninjured animals following treatment with rhIL-33 or PBS on Day 1 post injury. (F) miR-29a expression following the addition of anti IL-33 in post injured WT animals on days 1 and 3/Data are shown as the mean fold change±SD of duplicate samples and are representative of experiments using four mice per group (n=16) p<0.05, **p<0.01.

FIG. 6: IL-33/miR-29 axis in tendon pathology.

Schematic diagram illustrating the role of the IL-33/miR-29a in tendon pathology. An tendon injury or repetitive micro tears causing stress that a tendon cell experiences results in the release IL-33 and the downstream phosphorylation of NFkB which in turn represses miR-29a causing an increase in collagen type 3 and soluble ST2 production. An increase in collagen 3 reduces the tendons ultimate tensile strength lending it to early failure while soluble ST2 acts in an autocrine fashion which may ultimately be a protective mechanism whereby excess IL-33 is removed from the system.

FIG. 7

(A) Figure showing seed regions of the two Targetscan predicted miR-29a MRE sites: 29-1 and 29-2 (B) Luciferase activity in HEK 293 cells transfected with precursor miR-29 a/b/c (pre-miR-29) containing 3′UTR of Col 1 or Col 3. Activity was determined relative to controls transfected with scrambled RNA, which was defined as 100%. This was repeated in 3 independent experiments. * p<0.05, **p<0.01 versus scrambled control. (C) Cotransfection of HEK 293 cells with pre-miR-29a,b.c containing 3′UTR of soluble ST2 showing miR-29a significantly reducing the relative luciferase activity as compared with the scrambled RNA-transfected controls (n=3)

(D) The remaining miR-29 binding site present in the short col3a1 3′UTR variant was tested in a luciferase assay for its sensitivity to miR-29a and found to be fully active.

(E) Sequences of 3′RACE products of tenocyte collagen transcripts from human and horse. Polyadenylation signals are underlined. The miR29a MRE is shown in italics in the human Col3a1(short 3′UTR) transcript and the horse Col3a1 transcript.

FIG. 8

(A) Col3 mRNA, (B) Collagen 3 protein, (C) Coil mRNA and (D) Collagen 1 protein levels post treatment with miR-29a mimic after tendon injury in WT mice. Data for mRNA are total copy number of gene vs 18S housekeeping gene in duplicate samples. Data are mean±SD of duplicate samples, representative of 6 mice per group, *p<0.05, **p<0.01 vs control. (ANOVA)

### DETAILED DESCRIPTION OF THE INVENTION

### Materials and Methods

### Human Model of Tendinopathy

All procedures and protocols were approved by the Ethics Committee under ACEC No. 99/101. Fifteen supraspinatus tendon samples were collected from patients with rotator cuff tears undergoing shoulder surgery (Table 1). The mean age of the rotator cuff ruptured patients was 54 years (range, 35-70 years)—the mean tear size was 2.5 cm. Samples of the subscapularis tendon were also collected from the same patients. Patients were only included if there was no clinically detectable evidence of subscapularis tendinopathy on a preoperative MRI scan or macroscopic damage to the subscapularis tendon at the time of arthroscopy—by these criteria they represented a truly pre-clinical cohort. An independent control group was obtained comprising 10 samples of subscapularis tendon collected from patients undergoing arthroscopic surgery for shoulder stabilization without rotator cuff tears. The absence of rotator cuff tears was confirmed by arthroscopic examination. The mean age of the control group was 35 years (range, 20-41 years).

### Tissue Collection and Preparation

Arthroscopic repair of the rotator cuff was carried out using the standard three-portal technique as described previously described. The cross-sectional size of the rotator cuff tear was estimated and recorded as described previously³⁹. The subscapularis tendon was harvested arthroscopically from the superior border of the tendon 1 cm lateral to the glenoid labrum. The supraspinatus tendon was harvested from within 1.5 cm of the edge of the tear prior to surgical repair. For immunohistochemical staining the tissue samples were immediately fixed in 10% (v/v) formalin for 4 to 6 hours and then embedded in paraffin. Sections were cut to 5 μm thickness using a Leica-LM microtome (Leica Microsystems, Germany) and placed onto Superfrost Ultra Plus glass slides (Gerhard Menzel, Germany). The paraffin was removed from the tissue sections with xylene, rehydrated in graded alcohol and used for histological and immunohistochemical staining per previously established methodologies⁴⁰.

Human tendon derived cells were explanted from hamstring tendon tissue of 5 patients (age 18-30 years) undergoing hamstring tendon ACL reconstruction. Cultures were maintained at 37° C. in a humidified atmosphere of 5% CO₂ for 28 days. Cells were subcultured and trypinized at subconfluency, Cells from the 3^{rd} and 4^{th} passage were used in normoxic conditions.

### Histology and Immunohistochemistry Techniques

Human sections were stained with haematoxylin and eosin and toluidine blue for determination of the degree of tendinopathy as assessed by a modified version of the Bonar score⁴¹ (Grade 4=marked tendinopathy, Grade 3=advanced tendinopathy, 2=moderate degeneration 1=mild degeneration 0=normal tendon). This included the presence or absence of oedema and degeneration together with the degree of fibroblast cellularity and chondroid metaplasia. Thereafter, sections were stained with antibodies directed against the following markers:—IL-33 (Alexis, mouse monoclonal), ST2 (Sigma Aldrich, rabbit polyclonal), IL-1RaCP (ProSci, rabbit polyclonal) CD68 (pan macrophages), CD3 (T cells), CD4 (T Helper cells), CD206 (M₂ macrophages), and mast cell tryptase (mast cells) (Vector Labs).

Endogenous peroxidase activity was quenched with 3% (v/v) H₂O₂, and nonspecific antibody binding blocked with 2.5% horse serum in TBST buffer for 30 minutes. Antigen retrieval was performed in 0.01M citrate buffer for 20 minutes in a microwave. Sections were incubated with primary antibody in 2.5% (w/v) horse serum/human serum/TBST at 4° C. overnight. After two washes, slides were incubated with Vector ImmPRESS Reagent kit as per manufactures instructions for 30 minutes. The slides were washed and incubated with Vector ImmPACT DAB chromagen solution for 2 minutes, followed by extensive washing. Finally the sections were counterstained with hematoxylin. Positive (human tonsil tissue) and negative control specimens were included, in addition to the surgical specimens for each individual antibody staining technique. Omission of primary antibody and use of negative control isotypes confirmed the specificity of staining.

We applied a scoring system based on previous methods⁴² to quantify the immunohistochemical staining. Ten random high power fields (×400) were evaluated by three independent assessors (NLM, JHR, ALC). In each field the number of positive and negatively stained cells were counted and the percentage of positive cells calculated giving the following semi-quantitative grading; Grade 0=no staining, Grade 1=<10% cells stained positive, 2=10-20% cells stained positive, Grade 3=>20% cells positive.

Mouse sections were processed using the above protocol with antibodies directed against the following markers:—IL-33 (R&D systems, mouse monoclonal), ST2 (Sigma Aldrich, rabbit polyclonal), F4/80 (Serotec, mouse monoclonal) and Anti-Histamine (Sigma Aldrich, rabbit polyclonal).

### Matrix Regulation

Tenocytes were evaluated for immunocytochemical staining of collagen 1 and collagen 3 to assess tenocyte matrix production (Abcam). Total soluble collagen was measured from cell culture supernatants using the Sircol assay kit (Biocolor Ltd, Carrickfergus, Northern Ireland) according to the manufacturer's protocol. 1 ml of Sircol dye reagent was ded to 100 μl test sample and mixed for 30 min at room temperature. The collagen-dye complex was precipitated by centrifugation at 10,000×g for 10 min; and then washed twice with 500 μl of ethanol. The pellet was dissolved in 500 μl of alkali reagent. The absorbance was measured at 540 nm by microplate reader. The calibration curve was set up on the basis of collagen standard provided by the manufacturer. Additionally the concentration of human and mouse collagen 1 and 3 was assessed using ELISA with colour change measured at 450 nm by microplate reader along with standards supplier by the manufacturer (USCNK Life Science Inc).

### Signalling Experiments

Phosphorylation status of mitogen-activated protein kinases (MAPKs), extracellular signal regulated kinases (ERK1/2), c-Jun N-terminal kinases (JNKs) and p38 isoforms were evaluated using the Human Phospho-MAPK Array (R & D Systems Europe, UK) as per the manufacturer's instructions. The ERK inhibitor (FR180204) was purchased from CalbioChem (Merck KGaA, Germany) and used at IC₅₀=10 μM, a concentration previously determined to offer optimal specific inhibition relative to off target effects which was used previously in our laboratory⁴³.

Phosphorylation of NEKβ p65 was assessed using the InstantOne ELISA in cell lysates from treated and untreated tencocytes. The absorbance was measured at 450 nm by microplate reader with positive and negative controls supplied by the manufacturer. The relative absorbance of stimulated versus unstimulated cells was used to assess the total or phosphorylated NFKβ p65 in each sample.

### RNA Extraction and Quantitative PCR

The cells isolated from the normoxic and hypoxic experiments Trizol prior to mRNA extraction. QIAgen mini columns (Qiagen Ltd, Crawley UK) were used for the RNA clean-up with an incorporated on column DNAse step as per manufactures instructions. cDNA was prepared from RNA samples according to AffinityScript™ (Agilent Technologies, CA, USA) multiple temperature cDNA synthesis kit as per manufactures instructions. Real time PCR was performed using SYBR green or Tagman FastMix (Applied Biosystems, CA, USA) according to whether a probe was used with the primers. The cDNA was diluted 1 in 5 using RNase-free water. Each sample was analysed in triplicate. Primers (Integrated DNA Technologies, Belgium) were as follows: GAPDH, 5′-TCG ACA GTC AGC CGC ATC TTC TTT-3′ (f) and 5′-ACC AAA TCC GTT GAC TCC GAC CTT-3′ (r); IL-33 human GGA AGA ACA CAG CAA GCA AAG CCT (f) TAA GGC CAG AGC GGA GCT TCA TAA (r); IL-33 murine GGA AGA ACA CAG CAA GCA AAG CCT (f) TAA GGC CAG AGC GGA GCT TCA TAA (r); Total ST2 human ACA ACT GGA CAG CAC CTC TTG AGT (f) ACC TGC GTC CTC AGT CAT CAC ATT (r); sST2 murine CCA ATG TCC CTT GTA GTC GG (f) CTT GTT CTC CCC GCA GTC (r) TCC CCA TCT CCT CAC CTC CCT TAA T (probe); ST2L murine TCT GCT ATT CTG GAT ACT GCT TTC, TCT GTG GAG TAC TTT GTT CAC C (r) AGA GAC CTG TTA CCT GGG CAA GAT G (probe); human ST2L ACA AAG TGC TCT ACA CGA CTG (f) TGT TCT GGA TTG AGG CCA C (r); CCC CAT CTG TAC TGG ATT TGT AGT TCC G (probe); human sST2 GAG ACC TOO CAC GAT TAC AC (f) TGTTAAACCCTGAGTTCCCAC (r), CCC CAC ACC CCT ATC CTT TCT CCT (probe); Col 3A Human TTG GCA GCA ACG ACA CAG AAA CTG (f) TTG AGT GCA GGG TCA GCA CTA CTT (r) Col 3A Mouse GCT TTG TGC AAA GTG GAA CCT GG (f) CAA GGT GGC TGC ATC CCA ATT CAT (r); COL 1A1 Human CCA TGC TGC CCT TTC TGC TCC TTT (f) CAC TTG GGT GTT TGA GCA TTG CCT (r) COL 1A1 Mouse TTC TCC TGG CAA AGA CGG ACT CAA (f) GGA AGC TGA AGT CAT AAC CGC CA (r)

RNA Isolation and Quantitative Real Time PCR Analysis of miRNA

Total RNA was isolated by miRNeasy kit (Qiagen). miScript Reverse Transcription Kit (Qiagen) was used for cDNA preparation. TaqMan mRNA assays (Applied Biosystems) or miScript primer assay (Qiagen) were used for semi-quantitative determination of the expression of human miR-29a (MS (MS00001701) 29b (MS00006566) and c (MS00009303) and mouse 29a (MS00003262), 29b (MS00005936) and c (MS00001379). The expressions of U6B small nuclear RNA or beta-actin were used as endogenous controls.

### Quantification of Alternative Polyadenylated Collagen Transcripts

The absolute levels of long and short 3′UTR forms of type 1 and 3 transcripts were determined by q-PCR relative to standards. cDNA was generated using AffinityScript (Agilent) with both random hexamer and oligo-dT primers. SYBR green Quantitative-PCR was performed using the following primers: Samples were normalised to GAPDH endogenous control.

Col1a2_S FW 5′ GCCTGCCCTTCCTTGATATT 3′
Col1a2_S REV 5′ TGAAACAGACTGGGCCAATG 3′
col1a2_L FW 5′ TCAGATACTTGAAGAATGTTGATGG 3′
col1a2_L REV 5′ CACCACACGATACAACTCAATAC 3′
Col1a1_S FW 5′ CTTCACCTACAGCGTCACT 3′
Col1a1_S REV 5′ TTGTATTCAATCACTGTCTTGCC 3′
col1a1_L FW 5′ CCACGACAAAGCAGAAACATC 3′
col1al_L REV 5′ GCAACACAGTTACACAAGGAAC 3′
COL3A1_S FW 5′ CTATGACATTGGTGGTCCTGAT 3′
COL3A1_S REV 5′ TGGGATTTCAGATAGAGTTTGGT 3′
COL3A1_L FW 5′ CCACCAAATACAATTCAAATGC 3′
COL3A1_L REV 5′ GATGGGCTAGGATTCAAAGA 3′

3′ Rapid Extension of cDNA Ends (RACE)

To characterize human sequences, 3′RACE was performed on cDNA that had been generated from total RNA isolated from human tenocytes using MiRscript II reverse transcriptase kit (Qiagen). cDNA ends were amplified by PCR using the following gene specific forward primers listed below along with the Universal reverse primer from the kit.

Human 3′RACE gene specific forward primers:

RACE-Col1a1-L FW 5′ GACAACTTCCCAAAGCACAAAG 3′
RACE-Col1a1-S FW 5′ CTTCCTGTAAACTCCCTCCATC 3′
RACE-Col1a2-L FW 5′ TCTTCTTCCATGGTTCCACAG 3′
RACE-Col1a2-S FW 5′ CCTTCCTTGATATTGCACCTTTG 3′
RACE-Col3a1-L FW 5′ CTATGACATTGGTGGTCCTGAT 3′
RACE-Col3a1-S FW 5′ GTGTGACAAAAGCAGCCCCATA 3′

To characterise horse sequences, the 3′UTRs of Col1a1, Col1a2 and Col3a1 transcripts expressed in equine tenocytes were amplified using 3′ Rapid Extension of cDNA Ends (3′RACE). The amplified cDNA fragments were sequenced and the polyA signal identified according to the location of AATAAA canonical polyA signal located 10 and 30 nucleotides 5′ to the polyA tail.

Horse 3′RACE primers:

Horse col1a1 GSP1 CCCTGGAAACAGACAAACAAC
Horse col1a1 GSP2 CAGACAAACAACCCAAACTGAA
Horse col1a2 GSP1 GCTGACCAAGAATTCGGTTTG
Horse cola2 GSP2 ACATTGGCCCAGTCTGTTT
Horse col3a1 GSP1 AGGCCGTGAGACTACCTATT
Horse col3a1 GSP2 CTATGATGTTGGTGGTCCTGAT
Horse col1a1 q-PCR fw CAGACTGGCAACCTCAAGAA
Horse col1a1 q-PCR rev TAGGTGACGCTGTAGGTGAA
Horse col1a2 q-PCR fw GGCAACAGCAGGTTCACTTAT
Horse col1a2 q-PCR Rev GCAGGCGAGATGGCTTATTT
Horse col3a1 q-PCR fw CTGGAGGATGGTTGCACTAAA
Horse col3a1 q-PCR rev CACCAACATCATAGGGAGCAATA

The resulting PCR products were cloned into pCR2.1 TOPO (Invitrogen) and sequenced.

miRNA Transfection

Cells were transfected with synthetic mature miRNA for miR 29 a&b or with negative control (C. elegans miR-67 mimic labelled with Dy547, Thermo Scientific Inc) at a final concentration of 20 nM with the use of Dharmacon® DharmaFECT® 3 siRNA transfection reagents (Thermo Scientific Inc). At 48 hours after transfection cellular lysates were collected to analyse the expression of genes of interest.

Transfection efficiency was assessed by flow cytometry using the labelled Dy547 mimic and confirmed by quantitative PCR of control-scrambled mimic and the respective miR29 family mimic.

### Luciferase Reporter Assay for Targeting Collagen 1 & 3 and Soluble ST2

The human 2 miRNA target site was generated by annealing the oligos: for COL 1 & 3 and soluble ST2 3′UTR's which were cloned in both sense and anti-sense orientations downstream of the luciferase gene in pMIR-REPORT luciferase vector (Ambion). These constructs were sequenced to confirm inserts and named pMIR-COL I/COL III/sST2-miR29a/b/c and pMIR(A/S)-COL I/COL III/sST2-miR29a/b/c, and used for transfection of HEK293 cells. HEK293 cells were cultured in 96-well plates and transfected with 0.1 μg of either pMIR-COL I/COL III/sST2-miR29a/b/c, pMIR(A/S)-COL I/COL III/sST2-miR29a/b/c or pMIR-REPORT, together with 0.01 μg of pRL-TK vector (Promega) containing Renilla luciferase and 40 nM of miR-155 or scrambled miRNA (Thermo Scientific Dharmacon®). Transfections were done using Effectene (Qiagen) according manufacturer's instructions. Twenty-four hours after transfection, luciferase activity was measured using the Dual-Luciferase Reporter Assay (Promega). The 3′UTR of human sST2 was amplified from genomic DNA using the following primers sST2fw 5′AGTTTAAACTGGCTTGAGAAGGCACACCGT3′ and sST2rev 5′AGTCGACGGGCCAAGAAAGGCTCCCTGG3′ which created PmeI and SalI sites respectively. These sites where used to clone the PCR amplified product into the same sites of pmiRGLO (Promega). The seed regions of the two Targetscan predicted miR29a MRE sites: 29-1 and 29-2 were mutated using the QuickChange site-directed mutagenesis kit (Agilent). Each vector along with miR29a or scrambled control mimic were transfected into HEK293 cells using Attactene (Qiagen) according to manufactures instructions. After 24 hours luciferase activity was measured using Dual-Glo luciferase assay (Promega) with luciferase activity being normalized to Renilla. Normalized luciferase activity was expressed as a percentage of scrambled control for the same constructs.

### Cytokine Production

A 25-Plex human cytokine assay evaluated the in vitro quantitative determination of 25 separate human cytokines using Luminex technology. Supernatants (n=3)

### Patellar Tendon Injury Model

In preparation for the surgical procedure, mice were anesthetised with a mixture of isofluorane (3%) and oxygen (1%) and both hind limbs were shaved. During the surgical procedure, anaesthesia was delivered via a nose cone with the level of isofluorane reduced to 1% with the oxygen. Following a skin incision, two cuts parallel to the tendon were made in the retinaculum on each side, a set of flat faced scissors were then placed underneath the patellar tendon. With the scissor blades serving as a support, a 0.75 mm diameter biopsy punch (World Precision Instruments) was used to create a full thickness partial transection in the right patellar tendon. The left patellar tendon underwent a sham procedure, which consisted of only placing the plastic backing underneath the tendon without creating and injury. The skin wounds were closed with skin staples and the mice were sacrificed at 1 day, 3 days and 7 and 21 days post-surgery. Mice were sacrificed by CO₂ inhalation and immediately weighted. Mice from two groups BALB/c control (CTL) and ST2−/− BALB/c were used. Each group contained 16 mice (n=8 ST2−/− BALB/c and 8 BALB/c) per time point. These experiments were repeated on 4 separate occasions.

To test if IL-33 induced tendon matrix dysregulation a cytokine injection model was established. IL-33 was tested in a previously reported model initially described for the application of IL-23 or IL-22⁴⁴⁻⁴⁵ ST2−/− mice (n=4/group/treatment/experiment) were injected i.p. daily with IL-33 (0.2 μg per mouse diluted in 100 μL PBS) on days-3, -2, -1 and the day of injury. 24 hours following the final injection mice were culled as per protocol. Control mice similarly received an equal volume of PBS. We also tested neutralising antibodies to IL-33 (0.5 μg/ml R&D systems) by injecting i.p immediately post injury in WT and ST2−/− mice with IgG controls again with 4/group/treatment/experiment.

### Biomechanical Analysis

For the biomechanical analysis, the patellar tendons of mice from each group were injured and eight mice sacrificed at one of three time points for mechanical testing as described previously by Lin et al¹⁰. Briefly, the patellar tendons were dissected and cleaned, leaving only the patella, patellar tendon and tibia as one unit. Tendon width and thickness were then quantified and cross sectional area was calculated as the product of the two. The tibia was the embedded in Isopon p38 (High Build Cellulose Filler) in a custom designed fixture and secured in place in a metal clamp. The patella was held in place by vice grips used with the BOSE ElectroForce® 3200 test instrument. Each tendon specimen underwent the following protocol immersed in a 3700 saline bath—reloaded to 0.02N, preconditioned for 10 cycles from 0.02 to 0.04 at a rate of 0.1%/s (0.003 mm/s), and held for 10 s. Immediately following, a stress relaxation experiment was performed by elongating the tendon to a strain of 5% (0.015 mm) at a rate of 25% (0.75 mm/s), followed by a relaxation for 600 s. Finally a ramp to failure was applied at a rate of 0.1%/s (0.003 mm/s). From these tests, maximum stress was determined and modulus was calculated using linear regression from the near linear region of the stress strain curve.

In Vivo Administration of miR29a Mimic

A transfection complex was prepared containing 150 ng/ml miR-29a mimic, 9 μg/ml polyethylenimine (PEI) and 5% glucose. 50 μl of this complex was injected into mouse patellar tendon immediately after surgery. Animals were sacrificed after 1 and 3 days and col1a1 and col3a1 mRNA and protein levels were measured. Fluorescently labelled miR-29a mimic was used to assess the in vivo distribution of miR-29a mimic in the tendon by immunofluorescence, using counterstains for phalloidin (to show cytoskeletal structure) and nuclei (DAPI).

The miR29a mimic was as follows:

Passenger strand:

mAmCrCmGrAmUrUmUrCmArGmArUmGrGmUrGmCrUmAdG

Guide strand:

/5Phos/rUrArGrCrArCrCrArUrCrUrGrArArArUrCrGrGmUm
UmA

/5Phos/=5′ phosphate
mA=2′O-methyl adenosine ribonucleotide;
mC=2′O-methyl cytosine ribonucleotide;
mG=2′O-methyl guanine ribonucleotide;
mU=2′O-methyl uracil ribonucleotide;
rA=adenosine ribonucleotide;
rC=cytosine ribonucleotide;
rG=guanine ribonucleotide;
rU=uracil ribonucleotide;

### Statistical Analysis

All results are displayed as mean+/−standard error mean (SEM) and all statistical analysis was done either by students T test, ANOVA test or Mann Whitney test, as indicated in figure legends, using the Graph Pad Prism 5 software. A p value of <0.05 was considered statistically significant.

### Results

### IL-33 and ST2 Expression in Human Tendinopathy

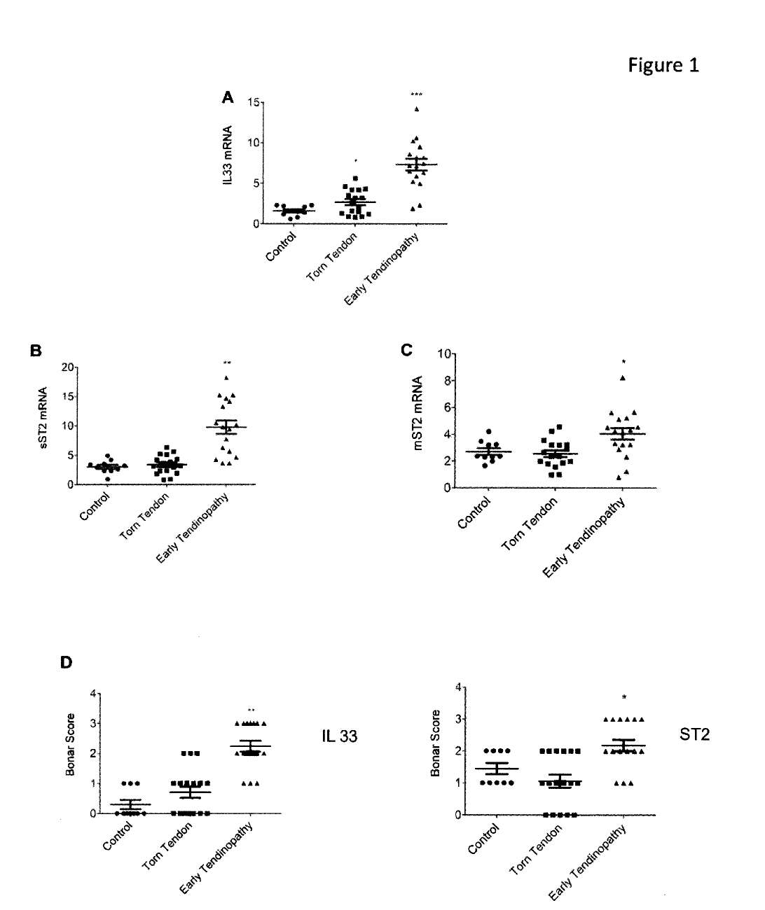

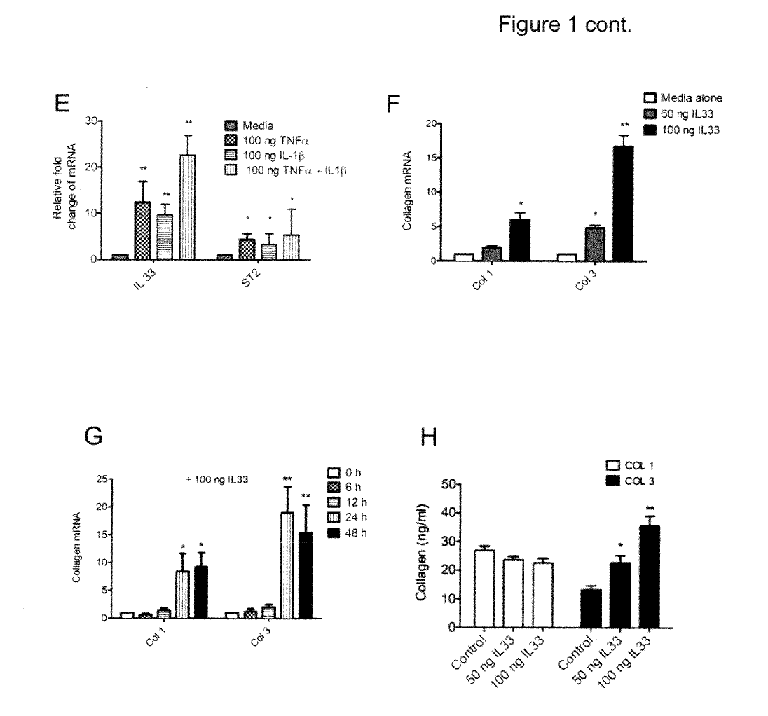

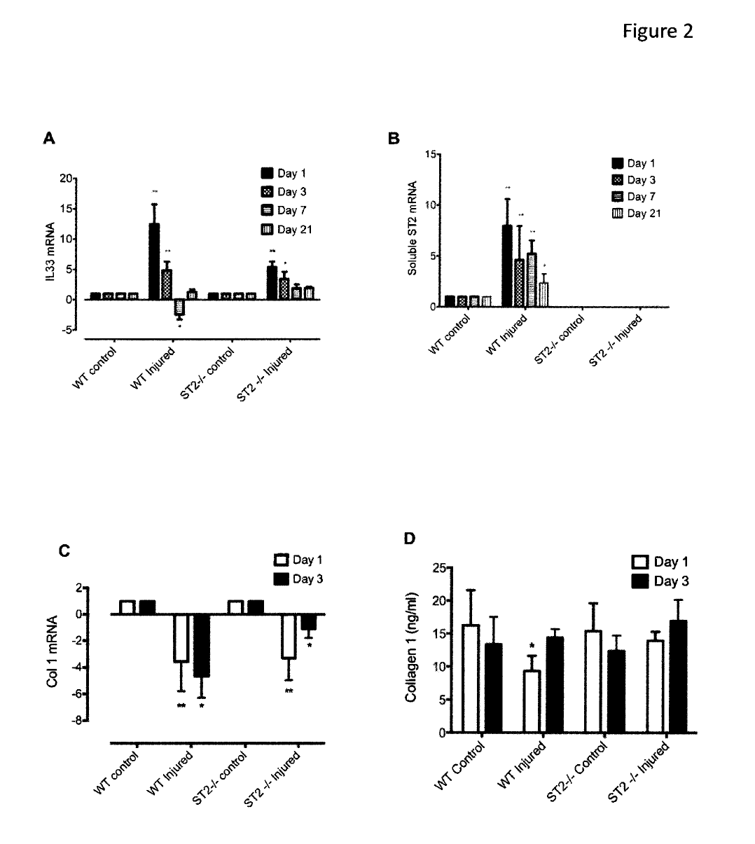

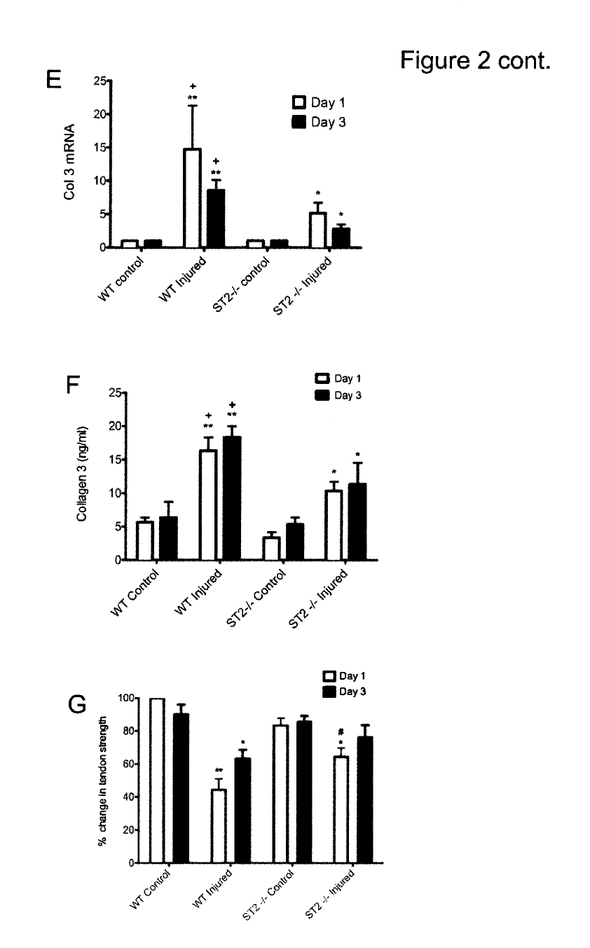

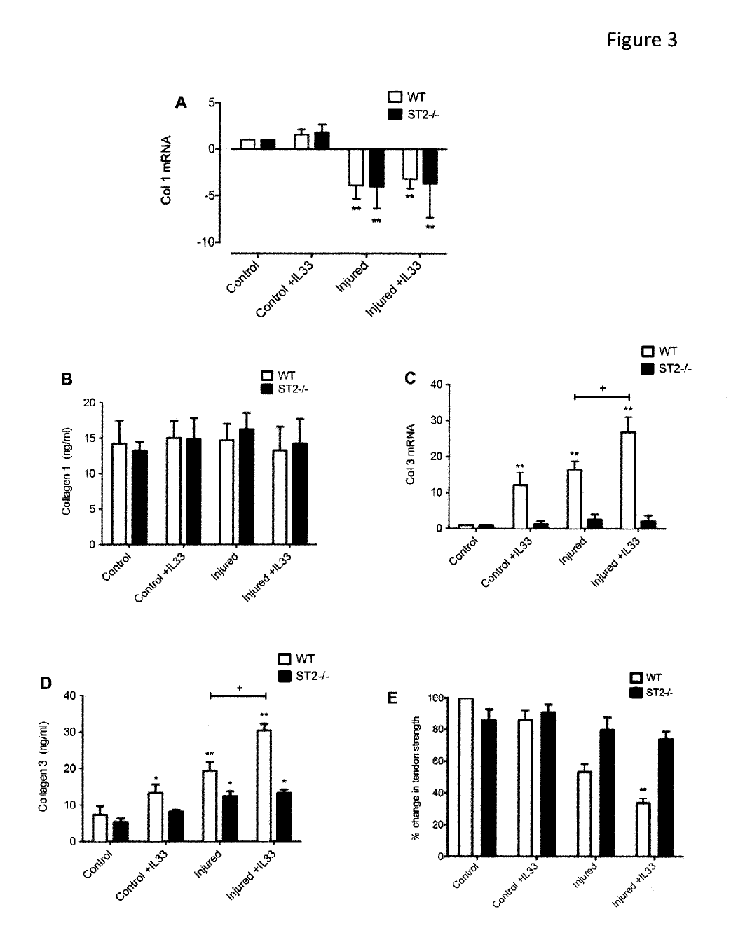

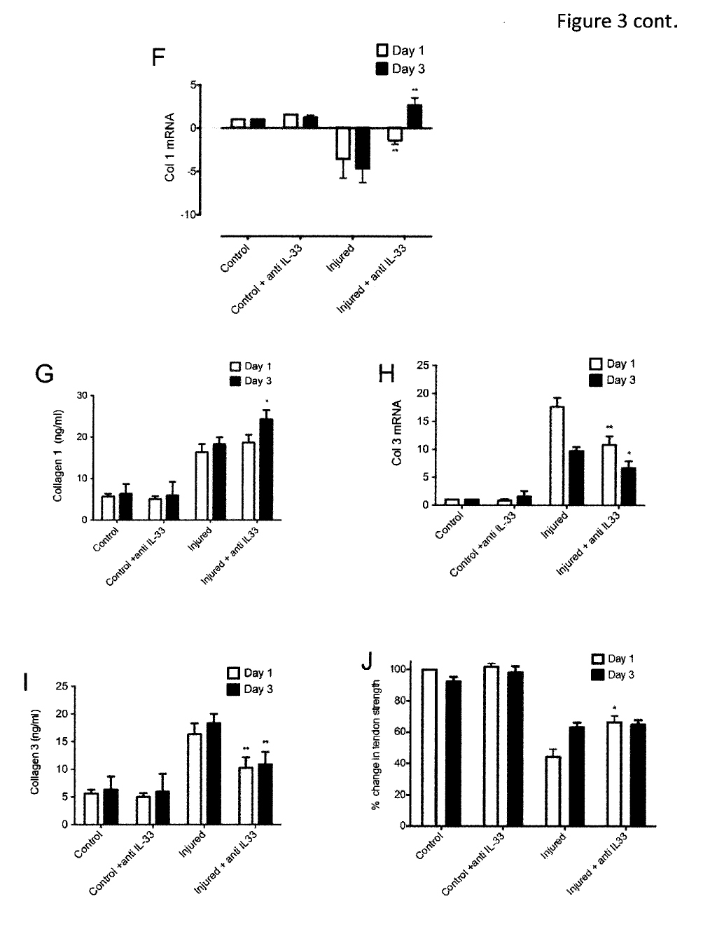

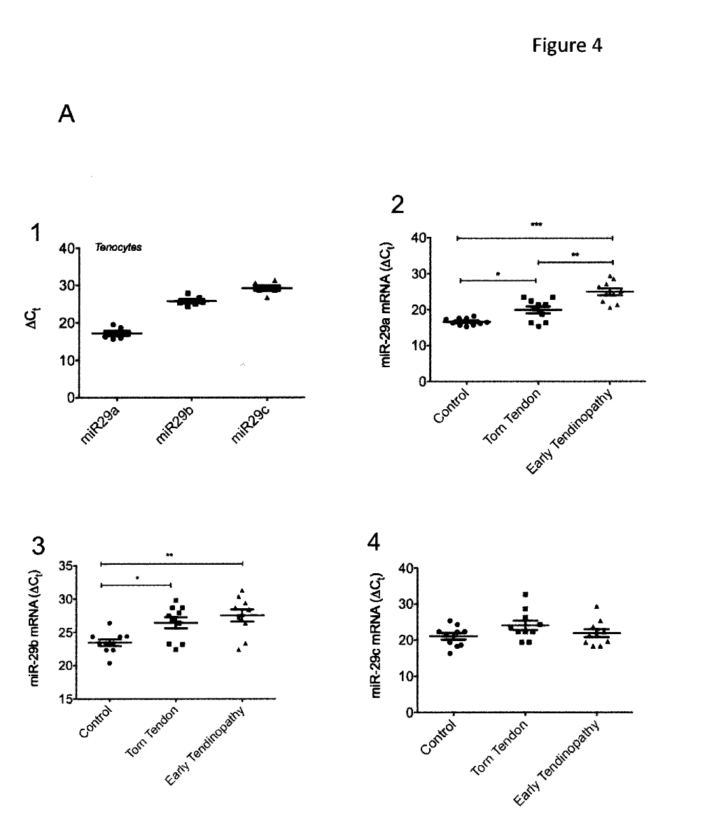

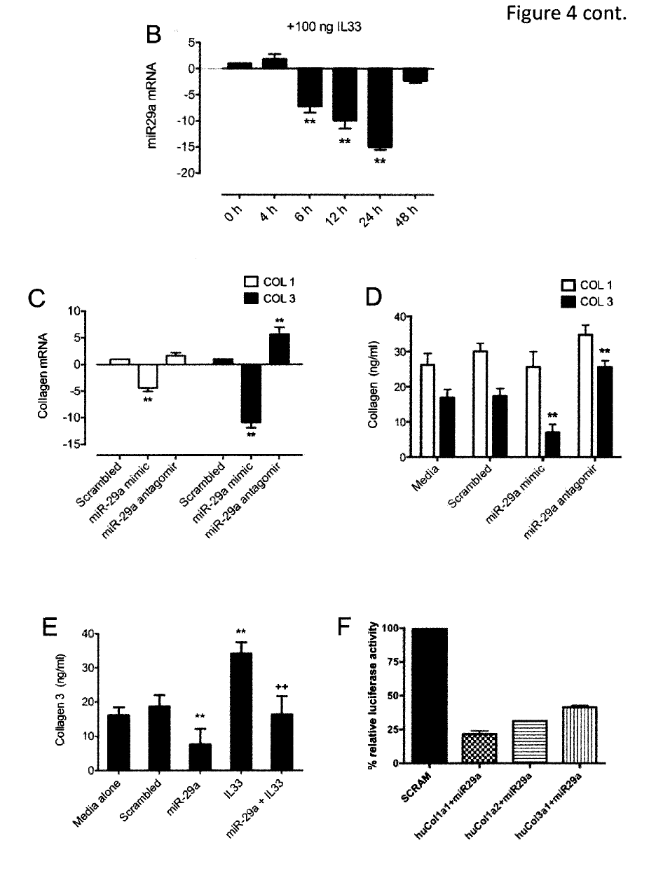

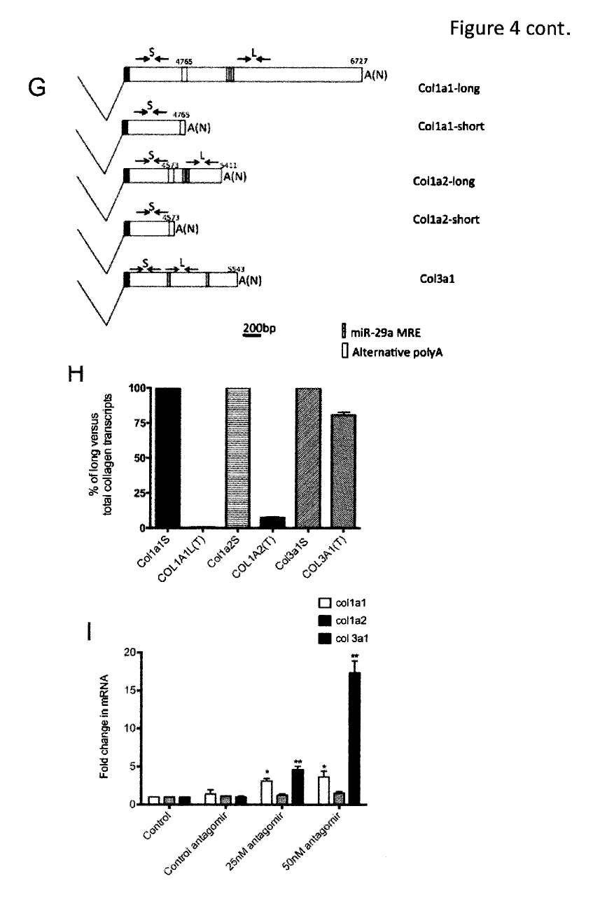

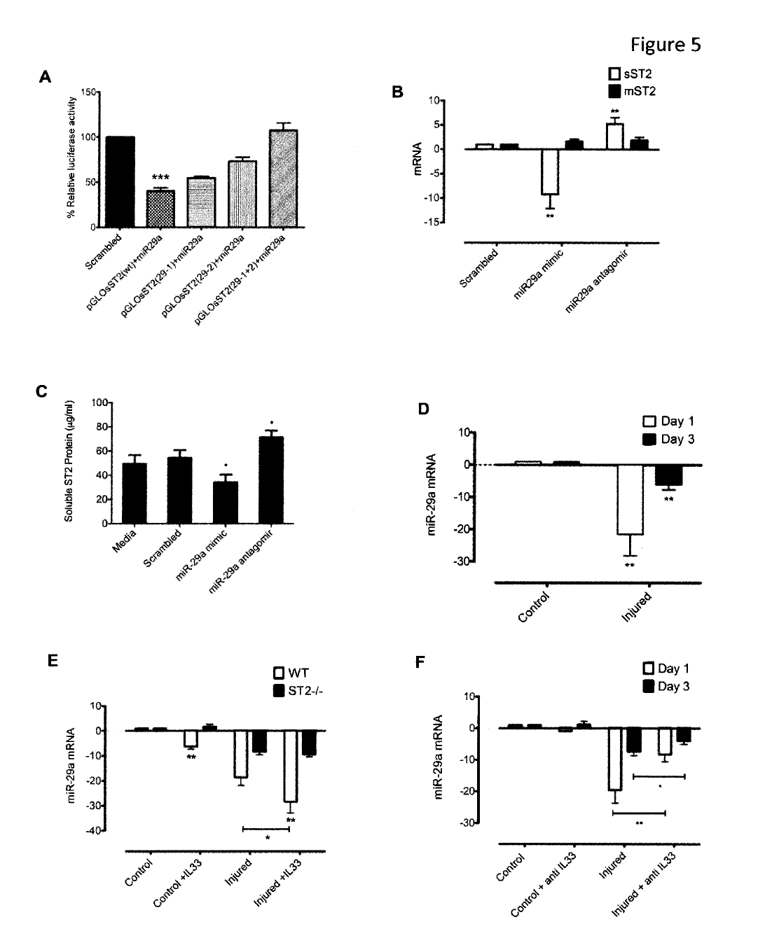

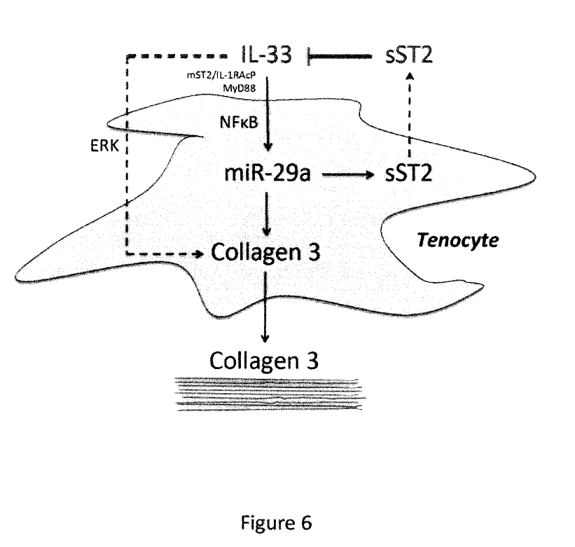

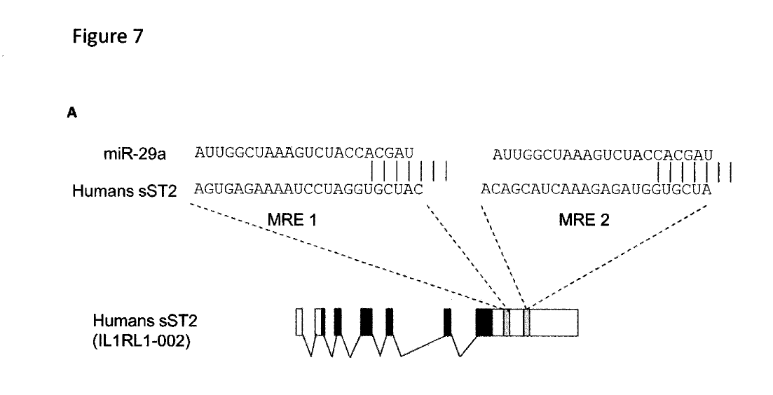

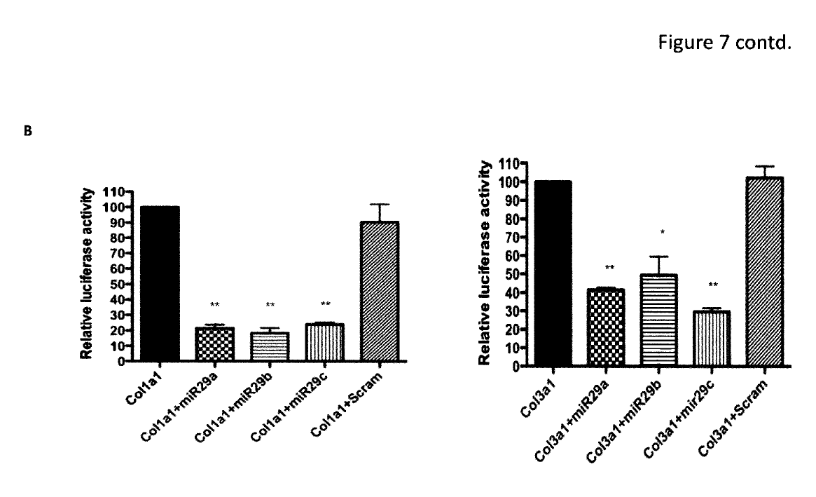

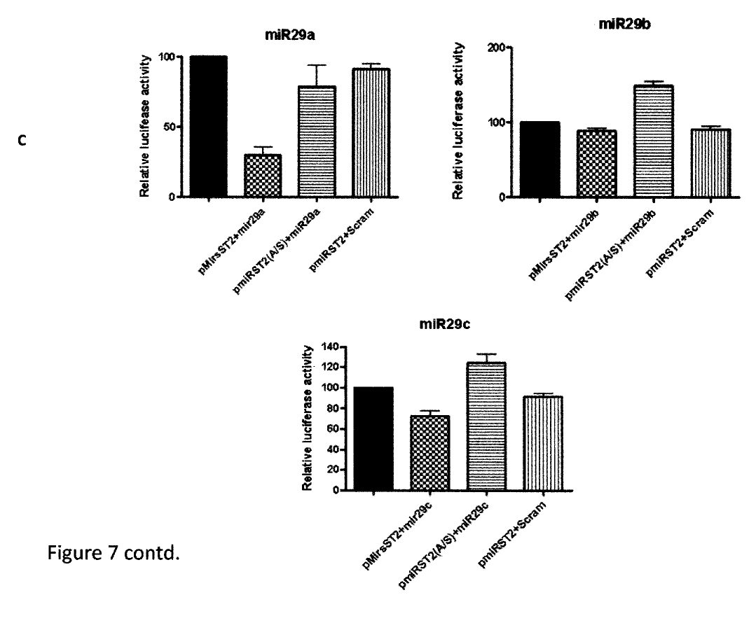

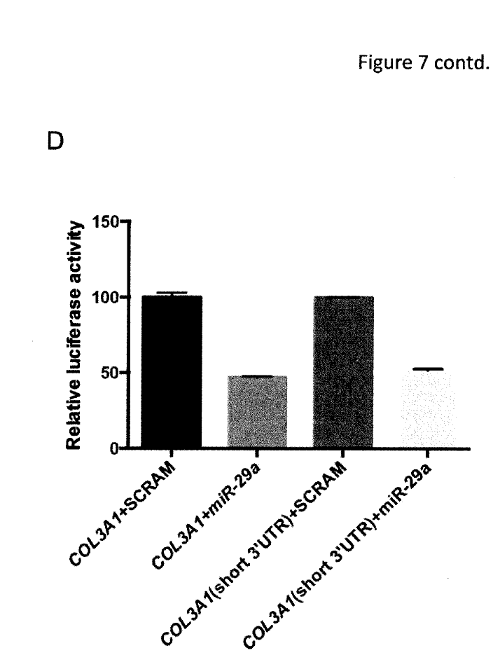

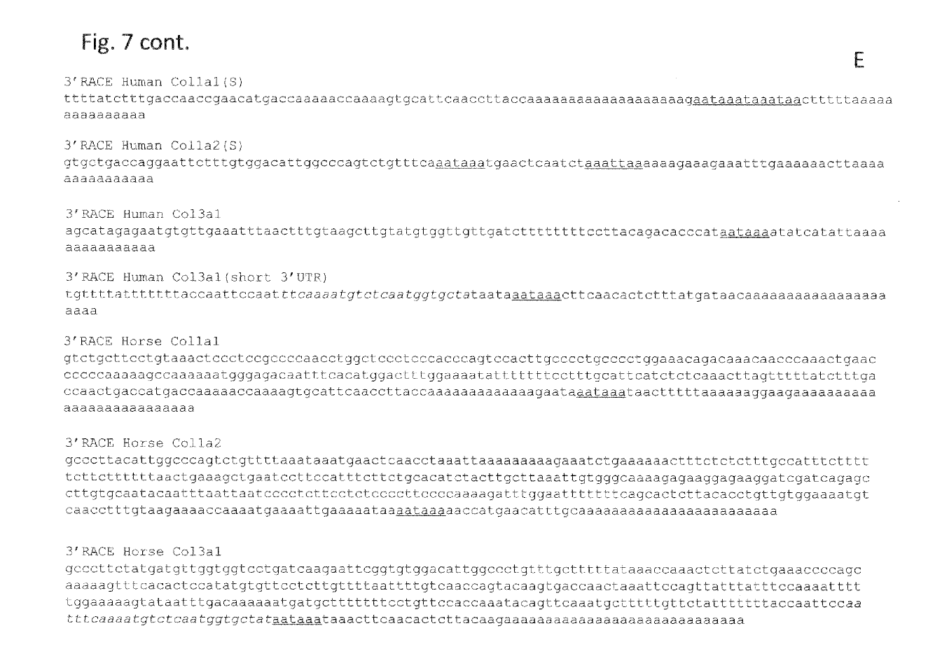

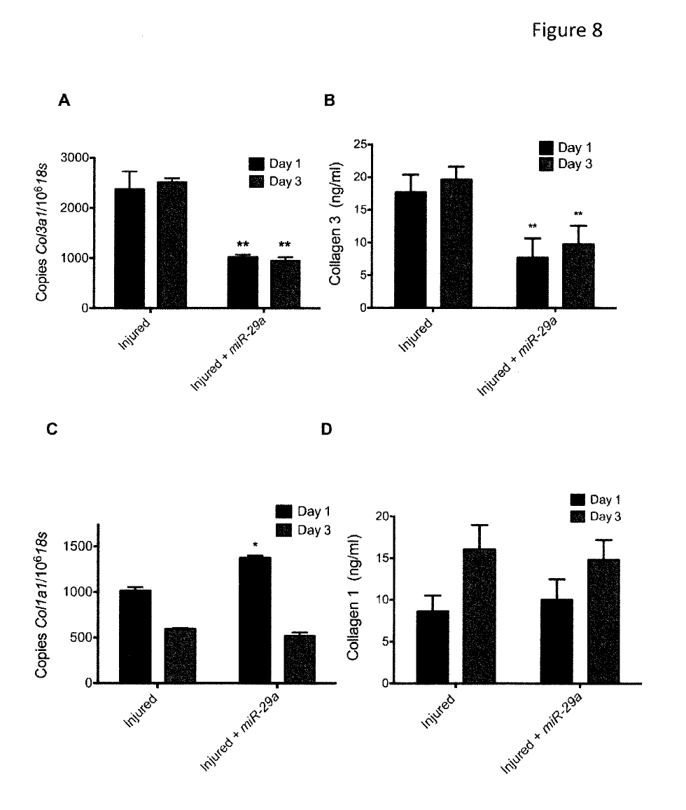

We first investigated IL-33 expression in human tendinopathy using our previously developed model²². IL-33, soluble and membrane bound ST2 transcripts were significantly upregulated in early tendinopathy compared to control or torn tendon biopsies (FIG. 1A-C). Early tendinopathy tissues exhibited significantly greater staining for IL-33 and ST2 compared to torn tendon or control biopsies (FIG. 1D). Staining was prominent in endothelial cells and particularly fibroblast-like cells, namely tenocytes that are considered pivotal to the regulation of early tendinopathy (data not shown). In parallel, in vitro cultured tenocytes expressed nuclear IL-33 that was up regulated at both mRNA and protein levels following stimulation by TNF and IL-1p (FIG. 1E and data not shown). In contrast ST2 was constitutively expressed in both resting and unstimulated tenocytes (data not shown).

### IL-33 Regulates Tenocyte Collagen Matrix and Proinflammatory Cytokine Synthesis

Matrix dysregulation towards collagen 3 expression is a key early phenotypic change in tendinopathy thereby hastening repair; collagen 3 is however biomechanically inferior. IL-33 induced dose and time dependent upregulation of total collagen protein (data not shown), accounted for by increased expression of type 1 but particularly type 3 collagen mRNA and protein (FIG. 1F, G). Following array analysis (data not shown) and consistent with reported IL-33 downstream signalling^{12,16}, this was abrogated by ERK inhibition (data not shown). rhIL-33 also significantly elevated production of IL-6, IL-8 and MCP-1 (data not shown), which was regulated by NF-kB inhibition suggesting that IL-33 operates in tenocytes via its canonical IL-1R signalling pathway (data not shown). In contrast we found no effect on production of other cytokines in keeping with previously reported IL-33 induced cytokine production profiles in fibroblasts²⁰⁻²³.

### Modelling IL-33/ST2 Pathway In Vivo Following Tendon Injury

We extended these observations to a well-established in vivo model of tendon injury. IL-33 mRNA was elevated on days 1 and 3 post tendon injury in WT mice (FIG. 2A). This was significantly reduced in injured ST2−/− mice suggesting autocrine regulation. Soluble ST2 was significantly up regulated at all time points post injury in WT mice compared to uninjured controls (FIG. 2B) whereas membrane ST2 mRNA was elevated only by Day 3 post injury (data not shown). No significant changes in IL-33 or ST2 transcript or protein expression were found in WT mice at days 7 or 21 post-injury, or for IL-33 expression in ST2−/− mice, suggesting that the impact of IL-33 expression is manifest early, in keeping with ‘alarmin’ type activity in tendon injury/repair.

Analysis of collagen synthesis revealed significantly greater expression of collagen 3 at all time points post injury in WT mice compared to uninjured controls or injured ST2−/− mice (FIGS. 2E, F & data not shown). Collagen 1 was initially down regulated (days 1, 3) at mRNA levels (FIG. 2C) in WT injured mice but reverted towards pre-injury levels by days 7 and 21 (data not shown) with a similar trend in collagen 1 protein expression (FIG. 2D). In contrast, ST2−/− injured mice showed prolonged reduction of collagen 1 synthesis (days 1, 3 & 7) returning to baseline only by day 21 (data not shown). Importantly injury of WT mice tendons resulted in a significant decrease in biomechanical strength at Day 1 post injury compared to ST2−/− (FIG. 2G) that recovered by days 7 and 21 (data not shown). These data suggest altered collagen matrix synthesis in ST2−/− mice implicating IL-33/ST2 as an early modulator of collagen changes in tendon injury that has biomechanical significance.

### Manipulating IL-33 Modifies Collagen 3 In Vivo

To confirm this possibility we sought to directly modify IL-33 effector biology in vivo. Administration of rhIL-33 did not affect collagen 1 synthesis (FIG. 3A,B) but did significantly increase collagen 3 synthesis particularly in injured tendons (FIG. 3D,E and data not shown). Moreover, rhIL-33 administration significantly reduced ultimate tendon strength at all time points post injection in WT mice (FIG. 3E and data not shown) suggesting that such changes were of functional impact. IL-33 administration did not affect collagen matrix synthesis or ultimate tendon strength of the healing tendon in ST2−/− mice confirming that IL-33 acted via an ST2-dependent pathway (data not shown).

We next directly targeted IL-33 in vivo. Neutralising antibodies to IL-33 attenuated the collagen 1 to 3 switch at days 1 and 3 post injury in WT injured mice (FIG. 3F-I) resulting in a significant increase in biomechanical strength at day 1 post injury WT mice tendons (FIG. 3J). This effect was not seen at later time points (data not shown). In control experiments we observed no effect on ST2−/− mice (data not shown) further confirming the contribution of endogenous IL-33 to injury-induced tendinopathy.

IL-33 Promotes Differential Regulation of Collagen 1/3 Via miR-29 in Tenocytes

Having established that IL-33 drives differential regulation of collagen 1 and 3 in tenocytes we postulated a mechanistic role for the miRNA network in this process. Previous studies have shown that the miR-29 family directly targets numerous extracellular matrix genes, including type 1 and 3 collagens²⁴⁻²⁵ and is implicated in regulation of innate and adaptive immunity²⁶. Computational algorithms predict that miR-29 may also target sST2. We found that all members of the miR-29 family were expressed in human tendon biopsies and explanted tenocytes (FIG. 4A) with miR-29a showing the most altered expression. In tenocyte culture IL-33 significantly reduced the expression of miR-29a at 6,12 and 24 hours (FIG. 4B) acting via NFκB dependent signalling whereas we observed inconsistent effects on miR-29b and c (data not shown). Since IL-33 mediated collagen 3 matrix changes could be regulated by miR-29a we analysed the functional effects of miR-29a manipulation on collagen matrix synthesis in vitro. Firstly, using luciferase assays, we confirmed that miR-29a directly targets col 1a1 and 3a1 as previously demonstrated²⁷ (FIG. 7B). We also observed a previously unrecognised interaction with col 1a2 subunit transcript (FIG. 7). To test whether miR-29a indeed regulates the levels of candidate target mRNAs in disease relevant cells, we transfected tenocytes with miR-29a mimic and antagomir. miR-29a manipulation selectively regulated collagen 3 but not collagen 1 mRNA and protein expression in primary tenocytes (FIG. 4C,D). Moreover, miR-29a over expression significantly abrogated IL-33 induced collagen 3 mRNA and protein synthesis (FIG. 4E). Additionally miR-29a inhibition resulted in a significant increase in col 3a1 expression indicating that miR-29a is not only actively regulating these transcripts in human tenocytes but whose loss is an important factor in the increase of type 3 collagen production observed in tendinopathy. In contrast col 1a1 transcript levels were unchanged (FIG. 4I).

Given that miR-29a was capable of repressing col 1a1 and 1a2 with equal or greater efficiency than collagen 3 in luciferase reporter assays, this was unlikely to be the result of miR-29a having greater affinity for its MREs in type 3 transcripts (FIG. 4F). One well-documented mechanistic explanation for transcripts to modulate their sensitivity to miRNA regulation is through the utilisation of alternative polyadenylation signals (FIG. 4G). To test this, we compared levels of full-length (miR-29a containing) transcripts to total levels by q-PCR (FIG. 4H) showing that in tenocytes, less than 5% of col 1a1 and 1a2 transcripts make use of the distal polyadenylation signal whereas the majority of col 3a1 transcripts do.

This was confirmed by 3′ rapid amplification of cDNA ends (RACE) (FIG. 7E) confirming that both col 1a1 and 1a2, but not col 3a1, make use of previously unrecognized polyadenylation signals (FIG. 4G). The resulting truncated 3′UTR lack miR29a MREs. (It will be appreciated that the sequences shown in FIG. 7E are cDNA sequences; the corresponding mRNA sequences would of course contain U rather than T.) These data suggest that in tenocytes, miR-29a specifically regulates col 3a1, while both col 1a1 and col 1a2 are rendered insensitive to miR-29a inhibition due to the utilisation of alternative polyadenylation signals. This utilisation of alternative polyadenylation signals was not influenced by the presence of IL-33 (data not shown). Loss of miR-29a upon IL-33 signalling results in depression of collagen 3 likely contributing to the increase of this collagen observed in injured tendons.

The 3′RACE results from human tenocytes revealed two col 3a1 UTRs, the shorter of which [designated Col3a1(short 3′UTR) in FIG. 7E] contains one miR-29a MRE, while the longer one contains two. Both are regulated by miR-29a as shown in FIG. 7D.

Characterisation of the 3′UTRs of Col1a1, Cola2 and Col3a1 transcripts expressed in equine tenocytes showed that they utilise the same conserved polyA signals used in the orthologous collagen transcripts expressed in human tenocytes. In col1a1 and cola2, use of these proximal polyA signals results in transcripts with 3′UTRs that are between 100 and 350 nucleotides in length and which do not contain miR-29 binding sites and therefore insensitive to regulation by this miRNA. In contrast both col3a1 3′UTRs contain miR-29 binding sites rendering them sensitive to regulation by miR-29.

Soluble ST2 is a Direct Target of miR-29

Computational analysis revealed that soluble ST2 can be targeted by miR-29a suggesting a feasible regulatory role in IL-33 effector functions. A luciferase reporter gene was generated that contains the 3′UTR of human sST2 predicted to possess two potential miR-29abc binding sites. Co-transfection of sST2-luciferase reporter plasmid with miR-29 mimics resulted in significant reduction in luciferase activity relative to scrambled control (FIG. 7B) Furthermore luciferase activity was fully restored when the seed regions of both miR-29 MREs in sST2 were mutated, demonstrating conclusively that sST2 is a direct target of miR-29a (FIG. 5A). To investigate whether miR-29a does indeed regulate the levels of the candidate target mRNA in tenocytes we again transfected miR-29a mimic and antagomir into human tenocytes. Soluble ST2 message was significantly (p<0.01) altered by transfection with miR-29a mimic/antagomir by approximately 5 fold (FIG. 5B) with a corresponding significant change in soluble ST2 protein confirming miR29a as a target for soluble ST2 (FIG. 5C).

IL-33/sST2 Regulates miR-29 Expression in In Vivo Models of Tendon Healing

Finally, we investigated miR-29a expression in our in vivo tendinopathy model. Tendon injury in WT mice resulted in a 22 fold decrease in miR29a on day 1 which reverted to a 6 fold decrease (versus baseline) by day 3 (FIG. 5D & data not shown) with no significant difference by day 7. This effect was significantly abrogated in ST2−/− mice (data not shown). In addition, administration of exogenous rh-IL-33 reduced miR-29a expression in uninjured tendons at all-time points compared to PBS injected controls (data not shown). This effect was most profound in injured WT mice, with the addition of rhIL-33 mediating a further 10 fold reduction in miR-29a (FIG. 5E). Addition of rhIL-33 in ST2−/− mice had no significant effect on miR-29a expression in injured or uninjured tendons again suggesting that miR-29a down regulation is in part directly mediated by IL-33/ST2 dependent signalling. The addition of neutralising antibody to IL-33 significantly reduced the effect of injury on miR-29a gene expression at days 1 and 3 post injury (FIG. 5F).

In Vivo Administration of miR29a Mimic in Patellar Tendon Injury Model

miR-29a mimic was delivered to tenocytes in WT mouse patellar tendons via direct injection of a miR-29a/PEI complex. Immunofluorescence staining for the mimic (red), counterstained with phalloidin (green, for cytoskeletal structure) and DAPI (to show nuclei) was used to visualise the localisation of mimic around tenocytes at 24 h post injection of miR-29a mimic (not shown). As shown in FIG. 8, collagen 3 mRNA and protein levels were significantly reduced in tendons injected with miR-29a mimic compared to controls. In contrast collagen 1 levels were unchanged.

### Preparation of Tendon-Derived ECM Scaffolds

### A. Preparation of Decellularized and Oxidized Tendon Scaffolds.

Freeze-dried human Achilles tendon allografts from multiple donors were provided and stored at 25° C. until use. Freeze-dried human Achilles tendon allografts were transferred under aseptic conditions to individual clean, autoclaved, 1000 ml glass flasks. 1000 ml of DNase-free/RNase-free, distilled water (Gibco) was added to each sample.

The flask was placed onto a rotating shaker (Barnstead MaxQ400, Dubuque, Iowa) at 200 rpm, 37° C., for 24 hours. After 24 hours, the water was discarded and the cycle was repeated. At the conclusion of the second cycle, the water was discarded and 500 ml of 0.05% trypsin-EDTA (Gibco) was added. The sample was placed onto the rotating shaker at 200 rpm, 37° C. for 1 hour. At the end of the cycle, the trypsin solution was discarded and 500 ml of Dulbecco's Modified Eagle's Medium (DMEM) high-glucose (Gibco) containing 10% fetal bovine serum (FBS) (Valley Labs, Winchester, Va.) and 100 I.U./ml Penicillin, 100 μg/ml Streptomycin, 0.25 ng/ml Amphotercin B (Gibco) was added in order to halt trypsin digestion of the sample.

The sample was placed back onto the rotary shaker at 200 rpm, 37° C., for 24 hours. After 24 hours, the DMEM-FBS solution was discarded and 1000 ml of the DNase-free/RNase-free distilled water was added and the sample was placed onto the rotary shaker at 200 rpm, 37° C. for 24 hours.

The water wash was discarded and 1000 ml of 1.5% peracetic acid (Sigma) solution with 1.5% Triton X-100 (Sigma) in distilled, deionized water was added and the sample placed onto the rotary shaker at 200 rpm, 37° C. for 4 hours. The solution was discarded and three 1000 ml washes with diH₂O were performed, each for 12 hours at 37° C. and 200 rpm on the rotary shaker. At the end of the third wash, the sample was removed and placed into a clean, sterile freezer bag and frozen for 24 hours at −80° C. The sample was then freeze-dried (Labconco, Freeze Dry System, Kansas City, Mo.) for 48 hours before being returned to the freezer and stored at −80° C. until further use.

### B. Histologic Analysis of Decellularized and Oxidized Tendon Scaffolds.

Mid-substance portions of freeze-dried human Achilles tendon allograft and decellularized and oxidized freeze-dried human Achilles tendon allograft-derived scaffold were placed in 10% phosphate-buffered formalin at room temperature for 4 hours. The tendons then were processed for histology, embedded in paraffin, and microtomed to obtain 5.0 μm thick, longitudinal sections. The sections were mounted on slides and stained using hematoxylin and eosin (H&E, Sigma) as well as 4′,6-diamidino-2-phenylindole (DAPI) (Vector, Burlingame, Calif.) to identify cellular and nuclear components, respectively. Representative light (H&E) and fluorescence (DAPI) micrographs were taken at 100× magnification. Abundant cellular material, specifically nuclear material, was evident after H&E and 4′,6-diamidino-2-phenylindole (DAPI) staining of longitudinal sections of freeze-dried human Achilles tendon allograft prior to decellularization and oxidation. Minimal porosity was observed in H&E stained sections of the freeze-dried human Achilles tendon allograft. After decellularization and oxidation, no nuclear material was evident via H&E staining. DAPI staining revealed the presence of DNA and RNA within the decellularized and oxidized tendon scaffolds. However, this material was neither organized, nor condensed in appearance as seen in the untreated tendons. An increase in intra-fascicular and inter-fascicular space after treatment was also observed via H&E staining.

### C. Determination of DNA Content in Decellularized and Oxidized Tendon Scaffolds.

Freeze-dried human Achilles tendon allograft (n=10) stored at −80° C. for 24 hours were lyophilized for 24 hours. Samples then were weighed and placed into sterile 1.5 ml micro-centrifuge tubes. This process was repeated for the decellularized and oxidized tendon scaffolds which previously had been freeze dried as part of their preparation process (n=8). Total DNA was then isolated from this tissue using a commercially available kit (DNeasy™, Qiagen, Valencia, Calif.). The DNA concentration in the resulting volume was used to calculate total DNA content at λ=280 nm using a spectrophotometer (Thermo Spectronic, Biomate 3, Rochester, N.Y.), which was then normalized using the initial dry weight of the sample.

DNA content of the decellularized and oxidized freeze-dried human Achilles tendon allograft-derived scaffolds was significantly decreased by 75% (0.110+/−0.02 μg DNA/mg tissue dry weight, n=10) after treatment when compared to untreated freeze-dried human Achilles tendon allografts (0.40+/−0.14 μg DNA/mg tissue dry weight, n=10), p<0.05.

### D. Transmission Electron Microscopy of Decellularized and Oxidized Tendon Scaffolds.

Transmission electron microscopy revealed that the decellularised and oxidised tendon scaffolds displayed a considerable decrease in fibril density per unit area as compared to the freeze-dried human Achilles tendon allograft, thus providing a scaffold having considerably increased pore size and porosity compared to the original allograft.

### E. In Vitro Biocompatibility of Decellularized and Oxidized Tendon Scaffolds: Direct Contact Method.

Representative specimens (approximately 0.04 cm³ portion/well) of the decellularized and oxidized freeze-dried human achilles tendon allograft-derived scaffolds (n=10) were placed in the center of sub-confluent murine NIH 3T3 cell monolayers in 96-well plates (Becton Dickinson), which covered one-tenth of the surface area, according to established standards (Pariente et al. (2001) J Biomed Mater Res 55:33-39). The same procedure was followed using latex (Ansell, Massillon, Ohio) as a negative control (n=10). Cells not exposed to any foreign material served as a positive control (n=10). The cell-material contact was maintained for 72 hours at 37° C. and 5% CO₂.

At the end of the incubation, the test materials were removed and two separate assays were performed to measure metabolic activity (MTS® solution) and cell viability (Neutral Red). Briefly, 40 μL of MTS solution (Promega, Madison, Wis.) was added into each well. After a 3 hour incubation at 37° C., the absorbance of the solution was measured at 490 nm using a 96-well plate spectrophotometer (Biotek, ELX800, Winoski, Vt.). The absorbance obtained was directly proportional to the metabolic activity of the cell populations and inversely proportional to the toxicity of the material.

For the cell viability assay, the media was removed and the cell layers rinsed with 200 μL, of cold PBS. 100 μL of neutral red solution (Sigma, 0.005% weight/volume in culture medium) was then added into each well. After a 3 hour incubation period at 37° C., the neutral red solution was removed and dye extraction performed by adding 100 μL of 1% (volume/volume) acetic acid in 50% (volume/volume) ethanol solution into each well. The plates were agitated on a platform shaker (Barnstead) for 5 minutes. Absorbance was measured at λ=540 nm using the 96-well plate spectrophotometer noted above. The absorbance obtained was directly proportional to the viability of the cell populations and inversely proportional to the toxicity of the material. The negative control (cells exposed to latex) for both assays was considered satisfactory if the observed absorbance for both assays was <10% of that observed for the positive control (cells exposed to media alone).

Mitochondrial activity determined using the MTS assay (absorbance at λ=490 nm) for NIH 3T3 cells exposed to the decellularized and oxidized freeze-dried human Achilles tendon allograft-derived scaffolds was 95% (1.36+/−0.31, n=10) of that observed for cells exposed to media only (1.42+/−0.31, n=10) a difference which was not statistically significant (p>0.05). Cell viability determined using the Neutral Red assay (absorbance at λ=540 nm) for NIH 3T3 cells exposed to the decellularized and oxidized freeze-dried human Achilles tendon allograft-derived scaffolds was 92% (0.24+/−0.07, n=10) of that observed for NIH 3T3 cells exposed to media alone (0.22+/−0.07, n=10, positive control), a difference which was not statistically significant. The decellularized and oxidized scaffold and positive control (cells only) differed significantly (p<0.001) from the values obtained for a known cytotoxic material (latex, negative control, n=10) in both assays. The absorbance observed for the negative control was also <10% of the absorbance observed for positive controls in each assay.

Preparation of Atelocollagen/Poly(Ethylene Glycol) Ether Tetrasuccinimidyl Glutarate Scaffold Impregnated with miR29a Mimic

Atelocollagen was isolated as described elsewhere⁵⁰. Nine parts of collagen solution (3.5 mg/ml w/v) was gently and thoroughly mixed with one part 10×PBS. The solution was neutralized by the drop-wise addition of 2 mol/l sodium hydroxide (NaOH) until a final pH of 7-7.5 was reached and kept in an ice bath to delay gel formation. 4S-StarPEG was then added at a final concentration of 0.125, 0.25, 0.5, and 1 mm in a volume of 200 μl as a cross-linking agent. 0.625% glutaraldehyde was used as a positive control. The solutions were incubated for 1 hour at 37° C. in a humidified atmosphere to induce gelation.

ECM derived biomaterials as delivery platforms of nonviral therapeutics have been previously documented both in vitro and in vivo, with beneficial outcomes. Previously, the in vitro effects of 4S-StarPEG crosslinked collagen type I scaffolds have been investigated as a delivery platform for delivering mesenchymal stem cells.

0.5 and 1 mmol concentrations of miR29a mimic will be added in a 5 ml volume to six well plates with 4S-StarPEG crosslinked collagen type I scaffolds and incubated for 2 hours. Functional PCR assays will be utilised to check saturation of the scaffold with miR29a mimic at this point.

Collagen scaffolds impregnated with miR29a mimic will be added to monolayer cultures of human and equine tenocytes and their effect on production of type I and III collagens (protein and mRNA) will be determined. Based on the results described above, a significant silencing of type III collagen in human and equine tenocytes is expected, with the balance of collagen synthesis being shifted in favour of type I collagen.

### Discussion

microRNAs have emerged as powerful regulators of diverse cellular processes with important roles in disease and tissue remodeling. These studies utilising tendinopathy as a model system reveal for the first time the ability of a single microRNA (miR-29) to cross regulate inflammatory cytokine effector function and extracellular matrix regulation in the complex early biological processes leading to tissue repair.

We herein provide new evidence for a role of IL-33 in the initial steps that lead to the important clinical entity of tendinopathy. IL-33 has recently become increasingly associated with musculoskeletal pathologies¹⁶. Our data show IL-33 to be present in human tendon biopsies at the early stage of disease while end stage biopsies have significantly less IL-33 expression at the message and protein level promoting the concept of IL-33 as an early tissue mediator in tendon injury and subsequent tissue remodelling. Upon cell injury endogenous danger signals, so called damage associated molecular patterns, are released by necrotic cells including heat shock proteins²⁸, HMGB1²⁹, uric acid³⁰ and IL-1 family members³¹⁻³² including IL-33³³⁻³⁴. These danger signals are subsequently recognised by various immune cells that initiate inflammatory and repair responses. Our data implicate IL-33 as an alarmin in early tendinopathy, and importantly, our biomechanical data suggest such expression has a pathogenically relevant role. The addition of rhIL-33 significantly reduced the load to failure of WT mice by approximately 30% at early time points, likely as a consequence of the concomitant collagen 3 matrix changes which result in mechanically inferior tendon³⁵. Thus one plausible mechanism for the events mediating early tendon repair that is biomechanically inferior, may be that upon repeated micro injury IL-33 is up regulated with its subsequent release through mechanical stress/necrosis, which in turn drives the matrix degeneration and proinflammatory cytokine production propelling the tendon toward a pathological state such as that seen in early tendinopathy biopsies. Interestingly the addition of neutralising antibodies to injured mice did reverse the collagen 3 phenotype but this was only able to temporarily improve tendon strength on day 1 post injury. Whilst this may negate blocking IL-33 in longer term sports injuries the repetitive microtrauma associated with pathological tendon changes may conversely allow neutralising IL-33 to act as a check rein to further unwanted matrix dysregulation.

Emerging studies highlight miRNAs as key regulators of leukocyte function and the cytokine network while orchestrating proliferation and differentiation of stromal lineages that determine extracellular matrix composition³⁶. The novel finding of a role for miR-29a in the regulation of IL-33 ‘alarmin’ mediated effects provides mechanistic insight into miRNA cross-regulatory networks involving inflammation and matrix regulation in tissue repair. Our data provide convincing evidence for a functional role for miR-29 as a posttranscriptional regulator of collagen in murine and human tendon injury. The regulation of collagens by the miR-29 family has been highlighted in several prior studies^{37 27}.³⁸. Our results now suggest that miR-29 acts as a critical repressor to regulate collagen expression in tendon healing. Moreover its reduced expression in human biopsies suggests that its functional diminution permissively permits development of tendinopathy. Despite tendon pathology being characterised by increased collagen 3 deposition resulting in biomechanical inferiority and degeneration the molecular premise for this collagen ‘switch’ has hitherto been unknown. We describe for the first time that IL-33 induced deficiency in miR-29a results in an over-production of collagen 3 whilst simultaneously setting in motion, via sST2 inhibition of IL-33, the ultimate resolution of this early repair process. Contrary to expectations in human tenocytes, miR-29 was only capable of influencing the expression of col 3a1 and not type 1 collagens. Subsequent characterisation of the 3′UTR of type 1 and 3 collagens revealed a previously unreported pattern of alternative polyadenylation in both type 1 subunits, resulting in transcripts lacking miR29a binding sites rendering them insensitive to repression by this miRNA. This was not the case for type 3 collagen transcripts, which retain both miR-29a binding sites. In human tenocytes, collagen 3 is actively repressed by miR-29a, as demonstrated by the ability of miR-29a inhibitors to significant increase collagen 3 levels while supplementing tenocytes with miR-29a in the presence of IL-33 was sufficient to inhibit the increase in collagen 3 production. Importantly in our model system miR-29a additionally targeted the IL-33 decoy receptor sST2. Thus IL-33 driven loss of miR-29a expression results in the simultaneous repression of collagen 3 and sST2, with a subsequent auto-regulatory inhibition of IL-33 promoting the resolution of the immediate alarmin response.

Based on this work we propose IL-33 as an influential alarmin in the unmet clinical area of early tendon injury and tendinopathy, which may be important in the balance between reparation and degeneration. A novel role for miR-29 as a posttranscriptional regulator of matrix/inflammatory genes in tendon healing and tendinopathy has been uncovered. One of the great promises of exploiting miRNAs for therapeutic purposes has been the potential of a single microRNA to regulate functionally convergent target genes. Our discovery of a single microRNA dependent regulatory pathway in early tissue healing, highlights miR-29 replacement therapy as a promising therapeutic option for tendinopathy with implications for many other human pathologies in which matrix dysregulation is implicated.

While the invention has been described in conjunction with the exemplary embodiments described above, many equivalent modifications and variations will be apparent to those skilled in the art when given this disclosure. Accordingly, the exemplary embodiments of the invention set forth are considered to be illustrative and not limiting. Various changes to the described embodiments may be made without departing from the spirit and scope of the invention. All documents cited herein are expressly incorporated by reference.

### REFERENCES

1. Eming, S. A., Krieg, T. & Davidson, J. M. Inflammation in wound repair: molecular and cellular mechanisms. J Invest Dermatol 127, 514-525 (2007).
2. McCormick, A., Charlton, J. & Fleming, D. Assessing health needs in primary care. Morbidity study from general practice provides another source of information. BMJ 310, 1534 (1995).
3. Nakama, L. H., King, K. B., Abrahamsson, S. & Rempel, D. M. Evidence of tendon microtears due to cyclical loading in an in vivo tendinopathy model. J Orthop Res 23, 1199-1205 (2005).
4. Sharma, P. & Maffulli, N. Tendon injury and tendinopathy: healing and repair. J Bone Joint Surg Am 87, 187-202 (2005).
5. Millar, N. L., Wei, A. Q., Molloy, T. J., Bonar, F. & Murrell, G. A. Cytokines and apoptosis in supraspinatus tendinopathy. J Bone Joint Surg Br 91, 417-424 (2009).
6. Pufe, T., Petersen, W., Tillmann, B. & Mentlein, R. The angiogenic peptide vascular endothelial growth factor is expressed in foetal and ruptured tendons. Virchows Arch 439, 579-585 (2001).
7. Tsuzaki, M., et al. IL-1 beta induces COX2, MMP-1, -3 and -13, ADAMTS-4, IL-1 beta and IL-6 in human tendon cells. J Orthop Res 21, 256-264 (2003).
8. Tohyama, H., Yasuda, K., Uchida, H. & Nishihira, J. The responses of extrinsic fibroblasts infiltrating the devitalised patellar tendon to IL-1beta are different from those of normal tendon fibroblasts. J Bone Joint Surg Br 89, 1261-1267 (2007).
9. John, T., et al. Effect of pro-inflammatory and immunoregulatory cytokines on human tenocytes. J Orthop Res 28, 1071-1077 (2010).
10. Lin, T. W., Cardenas, L., Glaser, D. L. & Soslowsky, L. J. Tendon healing in interleukin-4 and interleukin-6 knockout mice. J Biomech 39, 61-69 (2006).
11. Zhang, N. & Oppenheim, J. J. Crosstalk between chemokines and neuronal receptors bridges immune and nervous systems. J Leukoc Biot 78, 1210-1214 (2005).
12. Schmitz, J., et al. IL-33, an interleukin-1-like cytokine that signals via the IL-1 receptor-related protein ST2 and induces T helper type 2-associated cytokines. Immunity 23, 479-490 (2005).
13. Gao, P., Wange, R. L., Zhang, N., Oppenheim, J. J. & Howard, O. M. Negative regulation of CXCR4-mediated chemotaxis by the lipid phosphatase activity of tumor suppressor PTEN. Blood 106, 2619-2626 (2005).
14. Chen, X., Murakami, T., Oppenheim, J. J. & Howard, O. M. Triptolide, a constituent of immunosuppressive Chinese herbal medicine, is a potent suppressor of dendritic-cell maturation and trafficking. Blood 106, 2409-2416 (2005).
15. Lamkanfi, M. & Dixit, V. M. IL-33 raises alarm. Immunity 31, 5-7 (2009).
16. Liew, F. Y., Pitman, N. I. & McInnes, I. B. Disease-associated functions of IL-33: the new kid in the IL-1 family. Nat Rev Immunol 10, 103-110.
17. Asirvatham, A. J., Magner, W. J. & Tomasi, T. B. miRNA regulation of cytokine genes. Cytokine 45, 58-69 (2009).
18. Pritchard, C. C., Cheng, H. H. & Tewari, M. MicroRNA profiling: approaches and considerations. Nat Rev Genet 13, 358-369 (2012).
19. Matthews, T. J., Hand, G. C., Rees, J. L., Athanasou, N. A. & Carr, A. J. Pathology of the torn rotator cuff tendon. Reduction in potential for repair as tear size increases. J

Bone Joint Surg Br 88, 489-495 (2006).
20. Xu, D., et al. IL-33 exacerbates antigen-induced arthritis by activating mast cells. Proceedings of the National Academy of Sciences of the United States of America 105, 10913-10918 (2008).
21. Zaiss, M. M., et al. IL-33 shifts the balance from osteoclast to alternatively activated macrophage differentiation and protects from TNF-alpha-mediated bone loss. J Immunol 186, 6097-6105 (2011).
22. Rankin, A. L., et al. IL-33 induces IL-13-dependent cutaneous fibrosis. J Immunol 184, 1526-1535 (2010).
23. Palmer, G. & Gabay, C. Interleukin-33 biology with potential insights into human diseases. Nat Rev Rheumatol 7, 321-329.
24. Ogawa, T., et al. Suppression of type I collagen production by microRNA-29b in cultured human stellate cells. Biochem Biophys Res Commun 391, 316-321.
25. Bartel, D. P. MicroRNAs: target recognition and regulatory functions. Cell 136, 215-233 (2009).
26. Ma, F., et al. The microRNA miR-29 controls innate and adaptive immune responses to intracellular bacterial infection by targeting interferon-gamma. Nature immunology 12, 861-869 (2011).
27. Maurer, B., et al. MicroRNA-29, a key regulator of collagen expression in systemic sclerosis. Arthritis Rheum 62, 1733-1743 (2010).
28. Basu, S., Binder, R. J., Suto, R., Anderson, K. M. & Srivastava, P. K. Necrotic but not apoptotic cell death releases heat shock proteins, which deliver a partial maturation signal to dendritic cells and activate the NF-kappa B pathway. Int Immunol 12, 1539-1546 (2000).
29. Scaffidi, P., Misteli, T. & Bianchi, M. E. Release of chromatin protein HMGB1 by necrotic cells triggers inflammation. Nature 418, 191-195 (2002).
30. Shi, Y., Evans, J. E. & Rock, K. L. Molecular identification of a danger signal that alerts the immune system to dying cells. Nature 425, 516-521 (2003).
31. Chen, C. J., et al. Identification of a key pathway required for the sterile inflammatory response triggered by dying cells. Nat Med 13, 851-856 (2007).
32. Eigenbrod, T., Park, J. H., Harder, J., Iwakura, Y. & Nunez, G. Cutting edge: critical role for mesothelial cells in necrosis-induced inflammation through the recognition of IL-1 alpha released from dying cells. J Immunol 181, 8194-8198 (2008).
33. Moussion, C., Ortega, N. & Girard, J. P. The IL-1-like cytokine IL-33 is constitutively expressed in the nucleus of endothelial cells and epithelial cells in vivo: a novel ‘alarmin’? PLoS One 3, e3331 (2008).
34. Cayrol, C. & Girard, J. P. The IL-1-like cytokine IL-33 is inactivated after maturation by caspase-1. Proceedings of the National Academy of Sciences of the United States of America 106, 9021-9026 (2009).
35. James, R., Kesturu, G., Balian, G. & Chhabra, A. B. Tendon: biology, biomechanics, repair, growth factors, and evolving treatment options. J Hand Surg Am 33, 102-112 (2008).
36. Brown, B. D. & Naldini, L. Exploiting and antagonizing microRNA regulation for therapeutic and experimental applications. Nat Rev Genet 10, 578-585 (2009).
37. Roderburg, C., et al. Micro-RNA profiling reveals a role for miR-29 in human and murine liver fibrosis. Hepatology 53, 209-218 (2011).
38. Ogawa, T., et al. Suppression of type I collagen production by microRNA-29b in cultured human stellate cells. Biochem Biophys Res Commun 391, 316-321 (2010).
39. Millar, N. L., Wu, X., Tantau, R., Silverstone, E. & Murrell, G. A. Open versus two forms of arthroscopic rotator cuff repair. Clin Orthop Relat Res 467, 966-978 (2009).
40. McInnes, I. B., et al. Production of nitric oxide in the synovial membrane of rheumatoid and osteoarthritis patients. J Exp Med 184, 1519-1524 (1996).
41. Khan, K. M., Cook, J. L., Bonar, F., Harcourt, P. & Astrom, M. Histopathology of common tendinopathies. Update and implications for clinical management. Sports Med 27, 393-408 (1999).
42. Millar, N. L., Wei, A. Q., Molloy, T. J., Bonar, F. & Murrell, G. A. Heat shock protein and apoptosis in supraspinatus tendinopathy. Clin Orthop Relat Res 466, 1569-1576 (2008).
43. Kurowska-Stolarska, M., et al. IL-33 induces antigen-specific IL-5+ T cells and promotes allergic-induced airway inflammation independent of IL-4. J Immunol 181, 4780-4790 (2008).
44. Zheng, Y., et al. Interleukin-22, a T(H)17 cytokine, mediates IL-23-induced dermal inflammation and acanthosis. Nature 445, 648-651 (2007).
45. Hedrick, M. N., et al. CCR6 is required for IL-23-induced psoriasis-like inflammation in mice. The Journal of clinical investigation 119, 2317-2329 (2009).
46. Longo, U. G., Lamberti, A., Petrillo, S., Maffulli, N. & Denaro, V. Scaffolds in tendon tissue engineering. Stem Cells Int 2012, 517165 (2012).
47. Cooper, J. O., Bumgardner, J. D., Cole, J. A., Smith, R. A. & Haggard, W. O. Co-cultured tissue-specific scaffolds for tendon/bone interface engineering. J Tissue Eng 5, 2041731414542294 (2014).
48. Kim, B. S., et al. Human collagen-based multilayer scaffolds for tendon-to-bone interface tissue engineering. J Biomed Mater Res A 102, 4044-4054 (2014).
49. Woo, S. L. Tissue engineering: use of scaffolds for ligament and tendon healing and regeneration. Knee Surg Sports Traumatol Arthrosc 17, 559-560 (2009).
50. Zeugolis, DI, Paul, R G and Attenburrow, G (2008). Factors influencing the properties of reconstituted collagen fibers prior to self-assembly: animal species and collagen extraction method. J Biomed Mater Res A 86: 892-904.

1. A biocompatible implant comprising
(a) a biocompatible substrate capable of supporting growth of tendon cells; and
(b) a modulator of tendon healing;
wherein said modulator is
(i) miR-29, a mimic thereof, or a precursor of either; or
(ii) a nucleic acid encoding miR-29, a mimic thereof, or a precursor of either;
and wherein said modulator is located extracellularly to any cells present on or in said substrate.

2. A biocompatible implant according to claim 1 wherein the substrate is bioresorbable.

3. A biocompatible implant according to claim 1 or claim 2 wherein the substrate comprises one or more cells.

4. A biocompatible implant according to claim 3 wherein said cells comprise tenocytes, tenoblasts or mesenchymal stem cells.

5. A biocompatible implant according to any one of the preceding claims wherein the substrate is porous.

6. A biocompatible implant according to claim 5 wherein the substrate comprises a fabric, matrix, foam or gel.

7. A biocompatible implant according to claim 5 or claim 6 wherein the mean pore diameter is in the range of 10-500 μm, e.g. 50-500 μm, e.g. 100-500 μm or 200-500 μm.

8. A biocompatible implant according to any one of the preceding claims wherein the substrate comprises or consists of extra-cellular matrix (ECM).

9. A biocompatible implant according to claim 8 wherein the ECM is derived from tendon, small intestinal submucosa (SIS), dermis or pericardium.

10. A biocompatible implant according to claim 8 or claim 9 wherein said ECM has been subjected to decellularisation, oxidation, freeze drying, or any combination thereof.

11. A biocompatible implant according to any one of claims 1 to 10 wherein said ECM has been subjected to chemical cross-linking.

12. A biocompatible implant according to any one of claims 1 to 7 wherein the substrate is a synthetic substrate.

13. A biocompatible implant according to claim 12 wherein the substrate comprises one or more proteins or polysaccharides.

14. A biocompatible implant according to claim 13 wherein said proteins comprise one or more of collagen, elastin, fibrin, albumin and gelatin, and/or wherein said polysaccharides comprise one of more of hyaluronan, alginate and chitosan.

15. A biocompatible implant according to any one of claims 12 to 14 wherein said substrate comprises one or more synthetic polymers.

16. A biocompatible implant according to claim 15 wherein said synthetic polymer comprises one or more of polyvinyl alcohol, oligo[poly(ethylene glycol) fumarate] (OPF), poly(glycolic acid) (PGA), poly(lactic acid) (PLA), and poly(lactic-co-glycolic acid) (PLGA).

17. A biocompatible implant according to any one of claims 1 to 7 wherein the substrate comprises or consist of a bioceramic material or a biodegradable metallic material.

18. A biocompatible implant according to any one of the preceding claims wherein the substrate further comprises one or more cell adhesion peptides and/or one of more extracellular growth factors.

19. A biocompatible implant according to any one of the preceding claims wherein the modulator is a miR-29 mimic or precursor which comprises one or more modified sugar residues.

20. A biocompatible implant according to any one of the preceding claims wherein the modulator is a miR-29 mimic or precursor which comprises one or more modified internucleoside linkages.

21. A biocompatible implant according to any one of the preceding claims wherein the modulator is a miR-29 mimic or precursor which comprises one or more modified bases.

22. A biocompatible implant according to any one of the preceding claims wherein the modulator is a miR-29 mimic or precursor which comprises a membrane transit moiety.

23. A biocompatible implant according to any one of the preceding claims wherein the modulator is a miR-29, mimic, precursor or nucleic acid which is in association with (e.g. complexed with or encapsulated by) a carrier.

24. A biocompatible implant according to claim 23 wherein the carrier comprises a pharmaceutically acceptable lipid or polymer.

25. A biocompatible implant according to claim 23 or claim 24 wherein the carrier molecule comprises a targeting agent capable of binding to the surface of a target cell.

26. A biocompatible implant according to any one of claims 1 to 18 wherein the modulator is a nucleic acid which is comprised within a viral vector.

27. A biocompatible implant according to claim 26 wherein the viral vector is an adenovirus, adeno-associated virus (AAV), retrovirus or herpesvirus vector.

28. A biocompatible implant according to claim 27 wherein the retroviral vector is a lentiviral vector.

29. A biocompatible implant according to any one of the preceding claims wherein the miR-29 is miR-29a, miR-29b1, miR29b2 or miR-29c or a combination thereof.

30. A biocompatible implant according to claim 29 wherein the combination comprises miR-29a.

31. A biocompatible implant according to any one of the preceding claims wherein the modulator is or encodes a miR-29 or mimic thereof which comprises a guide strand comprising the seed sequence AGCACCA.

32. A biocompatible implant according to claim 31 wherein the guide strand comprises the sequence:
(hsa-miR-29a)
UAGCACCAUCUGAAAUCGGUUA;
(hsa-miR-29b1; ha-miR-29b2)
UAGCACCAUUUGAAAUCAGUGUU;
or
(ha-miR-29c)
UAGCACCAUUUGAAAUCGGUUA.

33. A biocompatible implant according to any one of the preceding claims wherein the modulator is or encodes a precursor which is pre-mir-29.

34. A biocompatible implant according to claim 33 wherein the pre-mir-29 comprises the sequence:
(hsa-pre-mir-29a: alternative (i))
AUGACUGAUUUCUUUUGGUGUUCAGAGUCAAUAUAAUUUUCUAGCACCAUC
UGAAAUCGGUUAU;
(hsa-pre-mir-29a: alternative (ii))
AUGACUGAUUUCUUUUGGUGUUCAGAGUCAAUAUAAUUUUCUAGCACCAUC
UGAAAUCGGUUAU AAUGAUUGGGG;
(hsa-pre-mir-29b1)
CUUCAGGAAGCUGGUUUCAUAUGGUGGUUUAGAUUUAAAUAGUGAUUGUCU
AGCACCAUUUGAAAUCAGUGUUCUUGGGGG;
(hsa-pre-mir-29b2)
CUUCUGGAAGCUGGUUUCACAUGGUGGCUUAGAUUUUUCCAUCUUUGUAUC
UAGCACCAUUUGAAAUCAGUGUUUUAGGAG;
or
(ha-pre-mir-29c)
AUCUCUUACACAGGCUGACCGAUUUGUCCUGGUGUUCAGAGUCUGUUUUUG
UCUAGCACCAUUUGAAAUCGGUUAUGAUGUAGGGGGA
(wherein the mature guide strand sequences are underlined).

35. A biocompatible implant according to any one of claims 1 to 32 wherein the modulator is or encodes a miR-29 mimic which comprises a guide strand comprising the sequence:
UAGCACCAUCUGAAAUCGGUUA (hsa-miR-29a);
UAGCACCAUUUGAAAUCAGUGUU (hsa-miR-29b1 and 2);
or
UAGCACCAUUUGAAAUCGCUUA (hsa-miR-29c)
(wherein the seed sequence is underlined in each case);
or which differs from said sequence at:
(i) no more than three positions within the seed sequence; and
(ii) no more than five positions outside the seed sequence.

36. A biocompatible implant according to any one of the preceding claims for use in a method of tendon therapy, e.g. in a method of surgery performed on a subject in need thereof.

37. A method of tendon therapy comprising locating a biocompatible implant according to any one of claims 1 to 35 at a site of injury.

38. Use of a modulator of tendon healing in the preparation of a biocompatible implant according to any one of claims 1 to 35, for use in a method of tendon therapy.

39. Use according to claim 38 wherein the modulator is incorporated into the substrate before the implant is introduced to a target site.

40. Use according to claim 38 wherein the substrate is introduced to a target site and the modulator subsequently applied to the substrate in situ.

41. A modulator of tendon healing for use in a method of tendon therapy;
wherein said method comprises applying said modulator to a biocompatible substrate capable of supporting growth of tendon cells;
wherein said biocompatible substrate is located at a site of tendon injury;
and wherein said modulator is:
(i) miR-29, a mimic thereof, or a precursor of either; or
(ii) a nucleic acid encoding miR-29, a mimic thereof, or a precursor of either.

42. Use of a modulator of tendon healing in the preparation of a pharmaceutically acceptable composition;
wherein said composition is for use in a method of tendon therapy which comprises applying said composition to a biocompatible substrate capable of supporting growth of tendon cells;
wherein said biocompatible substrate is located at a site of tendon injury;
and wherein said modulator is
(i) miR-29, a mimic thereof, or a precursor of either; or
(ii) a nucleic acid encoding miR-29, a mimic thereof, or a precursor of either.

43. A method of tendon therapy comprising locating a biocompatible substrate capable of supporting growth of tendon cells at a site of tendon injury, and applying a modulator of tendon healing to the biocompatible substrate,
wherein said modulator is
(i) miR-29, a mimic thereof, or a precursor of either; or
(ii) a nucleic acid encoding miR-29, a mimic thereof, or a precursor of either.

44. A kit comprising (a) a biocompatible substrate capable of supporting growth of tendon cells, and (b) a modulator of tendon healing, wherein said modulator is:
(i) miR-29, a mimic thereof, or a precursor of either; or
(ii) a nucleic acid encoding miR-29, a mimic thereof, or a precursor of either.


SEQUENCE LISTING (89 sequences)

SEQ ID NO: 1 - RNA, 22 nt, Homo sapiens
uagcaccaucugaaaucgguua

SEQ ID NO: 2 - RNA, 23 nt, Homo sapiens
uagcaccauuugaaaucaguguu

SEQ ID NO: 3 - RNA, 22 nt, Homo sapiens
uagcaccauuugaaaucgguua

SEQ ID NO: 4 - RNA, 22 nt, Homo sapiens
acugauuucuuuugguguucag

SEQ ID NO: 5 - RNA, 24 nt, Homo sapiens
gcugguuucauauggugguuuaga

SEQ ID NO: 6 - RNA, 22 nt, Homo sapiens
cugguuucacaugguggcuuag

SEQ ID NO: 7 - RNA, 22 nt, Homo sapiens
ugaccgauuucuccugguguuc

SEQ ID NO: 8 - RNA, 64 nt, Homo sapiens
augacugauuucuuuugguguucagagucaauauaauuuucuagcaccaucugaaaucgguuau

SEQ ID NO: 9 - RNA, 75 nt, Homo sapiens
augacugauuucuuuugguguucagagucaauauaauuuucuagcaccaucugaaaucgguuauaaugauugggg

SEQ ID NO: 10 - RNA, 81 nt, Homo sapiens
cuucaggaagcugguuucauauggugguuuagauuuaaauagugauugucuagcaccauuugaaaucaguguucuuggggg

SEQ ID NO: 11 - RNA, 81 nt, Homo sapiens
cuucuggaagcugguuucacaugguggcuuagauuuuuccaucuuuguaucuagcaccauuugaaaucaguguuuuaggag

SEQ ID NO: 12 - RNA, 88 nt, Homo sapiens
aucucuuacacaggcugaccgauuucuccugguguucagagucuguuuuugucuagcaccauuugaaaucgguuaugauguaggggga

SEQ ID NO: 13 - RNA, 1742 nt, Homo sapiens
gaaagcguuuuucuucaacuucuauggagcacuugcuugcuuuguccuauuugcauguccgacggacgguucuccagcaccacugcuagucguccuccgccugccuggguacuugaucacaggaugccucugacuucuccugccuuuacccaagcaaaggauuuuccuugucuucccacccaagagugacggggcugacaugugcccuugccucuaaaugaugaagcugaaccuuugucugggcaacuuaacuuaagaauaagggagucccaggcaugcucucccaucaauaacaaauucagugacaucaguuuaugaauauaugaaauuugccaaagcucuguuuagaccacugaguaacucacagcuagguuucaacuuuuccuuucuagguugucuuggguuuauuguaagagagcauuaugaagaaaaaaauagaucauaaagcuucuucaggaagcugguuucauauggugguuuagauuuaaauagugauugucuagcaccauuugaaaucaguguucuugggggagaccagcugcgcugcacuaccaacagcaaaagaagugaaugggacagcucugaaguauuugaaagcaacagcaggauggcugugagaaccugccucacauguagcugaccccuuccucaccccugccaacagugguggcauauaucacaaauggcagucaggucucugcacuggcggauccaacugugaucgaaaguuuuccaaaaauaaguugugucuguauugaacaugaacagacuuucuucuugucauuauucucuaacaauacugcauaacaauuauuugcauacauuugcauugcauuaaguauucuaaguaaucuagagacgauuuaaaguauacgggaggauguguguagguuguaugcaaauacuacaccauuuucuaucagagacuugagcaucuguggauuuugguauccaaggggcuuucuggaaccaaucccucaaggauaccaagggaugaauguaauuguacaggauaucgcauuguuggaauuuuauacuucuuuguggaauaaaccuauagcacuuaauagauaguacagacucauuccauugugccuggguuaaagagcccaauguaugcuggauuuaguaagauuugggcccucccaacccucacgaccuucugugaccccuuagaggaugacugauuucuuuugguguucagagucaauauaauuuucuagcaccaucugaaaucgguuauaaugauuggggaagagcaccaugaugcugacugcugagaggaaauguauuggugaccguuggggccauggacaagaacuaagaaaacaaaugcaaagcaauaaugcaaaggugauuuuucuucuuccaguuucuaaguugaauuucacugaccugaauugcaugugguauaauacuaacaaaugguucacuauuagcauaucaugaaugguuauacuuuauagaaauuccauagacuugguggggguuuuguuuuggugacggauaccuagaaacacuccuggggaaaaucgaugacuggcuuagaugaugggaaaggagcagcgagggagucaauucuguuguugaugagaagcugcaccagcuaucucugaacucuccucucuuagcuggcugaggaguucccuccaugguuaaacaggucauuuucuuacauaaggaaaaaugguccagagaaacuggguuucuauggcugagacagaacugugcuaauauguguc

SEQ ID NO: 14 - RNA, 2062 nt, Homo sapiens
agcuuucuaaaaucucuuuaggggugugcguaggcuccugugucuaugccugcuuuugacugcccaguugaagccucuuccuaugccuuuuaaaauuucacgcacuauaaggaggaagagcucagggcucccaaaacuuuuuauuuagagggaagaaugcuagggagauggguaugcagaggguugaccaaauuggaagaaaauauuuauucuguaguuugguguuggaaaagggaauuuuccaaucagccacaccucaguguugcggcaaaauaauucuuggcuccccuggaaacgcaugggcaagguagggcagagcugcugcugcugauacugccaccacccugggcuuccugcugacucugggcuacucccuggggacaacagauuugcauugacguccggggcuguccagaggcccucaagagccaguugugagcugagcccaguaugggaaagaucuaccuucuggaagcuacuacuacguggugcuuggaaagaggacucaggagagugcagcuugcucugugagugggugacaaccucuuggcgacucaggcucagcugaggauggugccagugugccggagacagccgucauacugccggauagaguggcucacuugcauguauuuggaacaaaaaaaggagaugccuggcagccccgcucucugcagugcuguugagccaccaauuuuugugguuuugugaccacaagugcugacugaugcgacaugaccccagucuugucagugaaucaucaccaggcugcuuacuggaaacuggaugcagcaaggaaauaggauuuaaccgcucucugccucccaggagcccugaaaucagcauucccagaggaaagaagauggccaucugggcuuggcuuccggcuccccccaucuggcuggaacacacaucagucaccccuguguaaccuccucugugccuuucccauggagcacugugucauaucacaaguagaacuacaagaagauauuucuccucagggcagaggcugggucuuccgauugaaucucccuucuuucuucauugagauccucuucuucuggaagcugguuucacaugguggcuuagauuuuuccaucuuuguaucuagcaccauuugaaaucaguguuuuaggaguaagaauugcagcacagccaaggguggacugcagaggaacugcugcucauggaacuggcuccucuccucuugccacuugagucuguucgagaaguccagggaagaacuugaagagcaaaauacacucuugaguuuguuggguuuugggagaggugacaguagagaaggggguuguguuuaaaauaaacacaguggcuugagcaggggcagagguugugaugcuauuucuguugacuccuagcagccaucaccagcaugaauguguucguagggccuuugaguguggcgauugucauauucuguuggauaacaauguauugggugucgauugucauggggcaggggagagggcaguacaccuggaggaccauuuuguccacaucgacaccaucagucugcucuuagaggaugcccuggaguauucggcguugauugcggggcacccgaaaucagacuugccaccuggacugucgaggugcagacccugggagcaccacuggcccaucucuuacacaggcugaccgauuucuccugguguucagagucuguuuuugucuagcaccauuugaaaucgguuaugauguagggggaaaagcagcagccucgaagccucaugccaacucugggcagcagcagccugugguuuccuggaagauggaugggcagagaauagggaaggaagaucaugcuuuucccuacuaacuucuguaacugcauguaugauacauuauugcagagguaagagauaguuuaauggauuuuuaaaaacaaauuacuauaauuuaucugauguucucuaguugcauuuugcugaaauguagugcuguucuaaauucuguaaauugauugcuguugaauuaucuuucuguugagaagagucuauucaugcauccugaccuuaauaaauacuauguucaguuu

SEQ ID NO: 15 - RNA, 23 nt, Homo sapiens
ccauuuuauaccaaaggugcuac

SEQ ID NO: 16 - RNA, 23 nt, Homo sapiens
uguucauaauacaaaggugcuaa

SEQ ID NO: 17 - RNA, 23 nt, Homo sapiens
uucaaaaugucucaauggugcua

SEQ ID NO: 18 - RNA, 22 nt, Artificial sequence
  Synthetic sequence miR-29 mimic guide strand
  modified_base (20)..(21): um
  misc_feature (22)..(22): 2prime O-methyl adenosine ribonucleotide
uagcaccaucugaaaucgguun

SEQ ID NO: 19 - RNA, 20 nt, Artificial sequence
  Synthetic sequence miR-29 mimic passenger strand
  misc_feature (1)..(1): 2prime O-methyl adenosine ribonucleotide
  modified_base (2)..(2): cm
  modified_base (4)..(4): gm
  modified_base (6)..(6): um
  modified_base (8)..(8): um
  misc_feature (10)..(10): 2prime O-methyl adenosine ribonucleotide
  misc_feature (12)..(12): 2prime O-methyl adenosine ribonucleotide
  modified_base (14)..(14): gm
  modified_base (16)..(16): um
  modified_base (18)..(18): cm
nccgauuucngnuggugcua

SEQ ID NO: 20 - DNA, 21 bp, Artificial sequence
  Synthetic sequence miR-29 mimic passenger strand
  misc_feature (1)..(1): 2prime O-methyl adenosine ribonucleotide
  modified_base (2)..(2): cm
  modified_base (4)..(4): gm
  modified_base (6)..(6): um
  modified_base (8)..(8): um
  misc_feature (10)..(10): 2prime O-methyl adenosine ribonucleotide
  misc_feature (12)..(12): 2prime O-methyl adenosine ribonucleotide
  modified_base (14)..(14): gm
  modified_base (16)..(16): um
  modified_base (18)..(18): cm
  misc_feature (20)..(20): 2prime O-methyl adenosine ribonucleotide
  misc_feature (21)..(21): deoxy-guanine
nccgauuucngnuggugcung

SEQ ID NO: 21 - DNA, 24 bp, Artificial sequence
  Synthetic primer
tcgacagtcagccgcatcttcttt

SEQ ID NO: 22 - DNA, 24 bp, Artificial sequence
  Synthetic primer
accaaatccgttgactccgacctt

SEQ ID NO: 23 - DNA, 24 bp, Artificial sequence
  Synthetic primer
ggaagaacacagcaagcaaagcct

SEQ ID NO: 24 - DNA, 24 bp, Artificial sequence
  Synthetic primer
taaggccagagcggagcttcataa

SEQ ID NO: 25 - DNA, 24 bp, Artificial sequence
  Synthetic primer
ggaagaacacagcaagcaaagcct

SEQ ID NO: 26 - DNA, 24 bp, Artificial sequence
  Synthetic primer
taaggccagagcggagcttcataa

SEQ ID NO: 27 - DNA, 24 bp, Artificial sequence
  Synthetic primer
acaactggacagcacctcttgagt

SEQ ID NO: 28 - DNA, 24 bp, Artificial sequence
  Synthetic primer
acctgcgtcctcagtcatcacatt

SEQ ID NO: 29 - DNA, 20 bp, Artificial sequence
  Synthetic primer
ccaatgtcccttgtagtcgg

SEQ ID NO: 30 - DNA, 18 bp, Artificial sequence
  Synthetic primer
cttgttctccccgcagtc

SEQ ID NO: 31 - DNA, 25 bp, Artificial sequence
  Synthetic probe
tccccatctcctcacctcccttaat

SEQ ID NO: 32 - DNA, 24 bp, Artificial sequence
  Synthetic primer
tctgctattctggatactgctttc

SEQ ID NO: 33 - DNA, 22 bp, Artificial sequence
  Synthetic primer
tctgtggagtactttgttcacc

SEQ ID NO: 34 - DNA, 25 bp, Artificial sequence
  Synthetic probe
agagacctgttacctgggcaagatg

SEQ ID NO: 35 - DNA, 21 bp, Artificial sequence
  Synthetic primer
acaaagtgctctacacgactg

SEQ ID NO: 36 - DNA, 19 bp, Artificial sequence
  Synthetic primer
tgttctggattgaggccac

SEQ ID NO: 37 - DNA, 28 bp, Artificial sequence
  Synthetic probe
ccccatctgtactggatttgtagttccg

SEQ ID NO: 38 - DNA, 20 bp, Artificial sequence
  Synthetic primer
gagacctgccacgattacac

SEQ ID NO: 39 - DNA, 21 bp, Artificial sequence
  Synthetic primer
tgttaaaccctgagttcccac

SEQ ID NO: 40 - DNA, 24 bp, Artificial sequence
  Synthetic probe
ccccacacccctatcctttctcct

SEQ ID NO: 41 - DNA, 24 bp, Artificial sequence
  Synthetic primer
ttggcagcaacgacacagaaactg

SEQ ID NO: 42 - DNA, 24 bp, Artificial sequence
  Synthetic primer
ttgagtgcagggtcagcactactt

SEQ ID NO: 43 - DNA, 23 bp, Artificial sequence
  Synthetic primer
gctttgtgcaaagtggaacctgg

SEQ ID NO: 44 - DNA, 24 bp, Artificial sequence
  Synthetic primer
caaggtggctgcatcccaattcat

SEQ ID NO: 45 - DNA, 24 bp, Artificial sequence
  Synthetic primer
ccatgctgccctttctgctccttt

SEQ ID NO: 46 - DNA, 24 bp, Artificial sequence
  Synthetic primer
cacttgggtgtttgagcattgcct

SEQ ID NO: 47 - DNA, 24 bp, Artificial sequence
  Synthetic primer
ttctcctggcaaagacggactcaa

SEQ ID NO: 48 - DNA, 23 bp, Artificial sequence
  Synthetic primer
ggaagctgaagtcataaccgcca

SEQ ID NO: 49 - DNA, 20 bp, Artificial sequence
  Synthetic primer
gcctgcccttccttgatatt

SEQ ID NO: 50 - DNA, 20 bp, Artificial sequence
  Synthetic primer
tgaaacagactgggccaatg

SEQ ID NO: 51 - DNA, 25 bp, Artificial sequence
  Synthetic primer
tcagatacttgaagaatgttgatgg

SEQ ID NO: 52 - DNA, 23 bp, Artificial sequence
  Synthetic primer
caccacacgatacaactcaatac

SEQ ID NO: 53 - DNA, 19 bp, Artificial sequence
  Synthetic primer
cttcacctacagcgtcact

SEQ ID NO: 54 - DNA, 23 bp, Artificial sequence
  Synthetic primer
ttgtattcaatcactgtcttgcc

SEQ ID NO: 55 - DNA, 21 bp, Artificial sequence
  Synthetic primer
ccacgacaaagcagaaacatc

SEQ ID NO: 56 - DNA, 22 bp, Artificial sequence
  Synthetic primer
gcaacacagttacacaaggaac

SEQ ID NO: 57 - DNA, 22 bp, Artificial sequence
  Synthetic primer
ctatgacattggtggtcctgat

SEQ ID NO: 58 - DNA, 23 bp, Artificial sequence
  Synthetic primer
tgggatttcagatagagtttggt

SEQ ID NO: 59 - DNA, 22 bp, Artificial sequence
  Synthetic primer
ccaccaaatacaattcaaatgc

SEQ ID NO: 60 - DNA, 20 bp, Artificial sequence
  Synthetic primer
gatgggctaggattcaaaga

SEQ ID NO: 61 - DNA, 22 bp, Artificial sequence
  Synthetic primer
gacaacttcccaaagcacaaag

SEQ ID NO: 62 - DNA, 22 bp, Artificial sequence
  Synthetic primer
cttcctgtaaactccctccatc

SEQ ID NO: 63 - DNA, 21 bp, Artificial sequence
  Synthetic primer
tcttcttccatggttccacag

SEQ ID NO: 64 - DNA, 23 bp, Artificial sequence
  Synthetic primer
ccttccttgatattgcacctttg

SEQ ID NO: 65 - DNA, 22 bp, Artificial sequence
  Synthetic primer
ctatgacattggtggtcctgat

SEQ ID NO: 66 - DNA, 22 bp, Artificial sequence
  Synthetic primer
gtgtgacaaaagcagccccata

SEQ ID NO: 67 - DNA, 21 bp, Artificial sequence
  Synthetic primer
ccctggaaacagacaaacaac

SEQ ID NO: 68 - DNA, 22 bp, Artificial sequence
  Synthetic primer
cagacaaacaacccaaactgaa

SEQ ID NO: 69 - DNA, 21 bp, Artificial sequence
  Synthetic primer
gctgaccaagaattcggtttg

SEQ ID NO: 70 - DNA, 19 bp, Artificial sequence
  Synthetic primer
acattggcccagtctgttt

SEQ ID NO: 71 - DNA, 20 bp, Artificial sequence
  Synthetic primer
aggccgtgagactacctatt

SEQ ID NO: 72 - DNA, 22 bp, Artificial sequence
  Synthetic primer
ctatgatgttggtggtcctgat

SEQ ID NO: 73 - DNA, 20 bp, Artificial sequence
  Synthetic primer
cagactggcaacctcaagaa

SEQ ID NO: 74 - DNA, 20 bp, Artificial sequence
  Synthetic primer
taggtgacgctgtaggtgaa

SEQ ID NO: 75 - DNA, 21 bp, Artificial sequence
  Synthetic primer
ggcaacagcaggttcacttat

SEQ ID NO: 76 - DNA, 20 bp, Artificial sequence
  Synthetic primer
gcaggcgagatggcttattt

SEQ ID NO: 77 - DNA, 21 bp, Artificial sequence
  Synthetic primer
ctggaggatggttgcactaaa

SEQ ID NO: 78 - DNA, 23 bp, Artificial sequence
  Synthetic primer
caccaacatcatagggagcaata

SEQ ID NO: 79 - DNA, 30 bp, Artificial sequence
  Synthetic primer
agtttaaactggcttgagaaggcacaccgt

SEQ ID NO: 80 - DNA, 28 bp, Artificial sequence
  Synthetic primer
agtcgacgggccaagaaaggctccctgg

SEQ ID NO: 81 - RNA, 23 nt, Homo sapiens
agugagaaaauccuaggugcuac

SEQ ID NO: 82 - RNA, 23 nt, Homo sapiens
acagcaucaaagagauggugcua

SEQ ID NO: 83 - DNA, 110 bp, Homo sapiens
ttttatctttgaccaaccgaacatgaccaaaaaccaaaagtgcattcaaccttaccaaaaaaaaaaaaaaaaaaagaataaataaataactttttaaaaaaaaaaaaaaa

SEQ ID NO: 84 - DNA, 110 bp, Homo sapiens
gtgctgaccaggaattctttgtggacattggcccagtctgtttcaaataaatgaactcaatctaaattaaaaaagaaagaaatttgaaaaaacttaaaaaaaaaaaaaaa

SEQ ID NO: 85 - DNA, 110 bp, Homo sapiens
agcatagagaatgtgttgaaatttaactttgtaagcttgtatgtggttgttgatcttttttttccttacagacacccataataaaatatcatattaaaaaaaaaaaaaaa

SEQ ID NO: 86 - DNA, 103 bp, Homo sapiens
tgttttatttttttaccaattccaatttcaaaatgtctcaatggtgctataataaataaacttcaacactctttatgataacaaaaaaaaaaaaaaaaaaaaa

SEQ ID NO: 87 - DNA, 310 bp, Equus caballus
gtctgcttcctgtaaactccctccgccccaacctggctccctcccacccagtccacttgcccctgcccctggaaacagacaaacaacccaaactgaaccccccaaaaagccaaaaaatgggagacaatttcacatggactttggaaaatatttttttcctttgcattcatctctcaaacttagtttttatctttgaccaactgaccatgaccaaaaaccaaaagtgcattcaaccttaccaaaaaaaaaaaaagaataaataaataactttttaaaaaaggaagaaaaaaaaaaaaaaaaaaaaaaaaaa

SEQ ID NO: 88 - DNA, 377 bp, Equus caballus
gcccttacattggcccagtctgttttaaataaatgaactcaacctaaattaaaaaaaaagaaatctgaaaaaactttctctctttgccatttctttttcttcttttttaactgaaagctgaatccttccatttcttctgcacatctacttgcttaaattgtgggcaaaagagaaggagaaggatcgatcagagccttgtgcaatacaatttaattaatcccctcttcctctccccttccccaaaagatttggaatttttttcagcactcttacacctgttgtggaaaatgtcaacctttgtaagaaaaccaaaatgaaaattgaaaaataaaataaaaaccatgaacatttgcaaaaaaaaaaaaaaaaaaaaaaaa

SEQ ID NO: 89 - DNA, 370 bp, Equus caballus
gcccttctatgatgttggtggtcctgatcaagaattcggtgtggacattggccctgtttgctttttataaaccaaactcttatctgaaaccccagcaaaaagtttcacactccatatgtgttcctcttgttttaattttgtcaaccagtacaagtgaccaactaaattccagttatttatttccaaaatttttggaaaaagtataatttgacaaaaaatgatgctttttttcctgttccaccaaatacagttcaaatgctttttgttctatttttttaccaattccaatttcaaaatgtctcaatggtgctataataaataaacttcaacactcttacaagaaaaaaaaaaaaaaaaaaaaaaaaaaaaa